(19) United States
(12) Patent Application Publication
(10) Pub. No.: US 2015/0140073 A1
(43) Pub. Date: May 21, 2015

(54) Heparosan-Multimolecular Assembly Drug Delivery Compositions and Methods of Making and Using Same

(71) Applicant: The Board of Regents of the University of Oklahoma, Norman, OK (US)

(72) Inventor: Paul L. DeAngelis, Edmond, OK (US)

(21) Appl. No.: 14/607,893

(22) Filed: Jan. 28, 2015

Related U.S. Application Data
(63) Continuation-in-part of application No. 13/715,117, filed on Dec. 14, 2012.
Application No. 13/715,117 is a division of application No. 12/556,324, filed on Sep. 9, 2009.
Application No. 12/556,324 is a continuation-in-part of application No. 12/383,046, filed on Mar. 19, 2009, now abandoned.
(60) Provisional application No. 61/932,570, filed on Jan. 28, 2014.
(60) Provisional application No. 61/179,275, filed on May 18, 2009.
(60) Provisional application No. 61/095,572, filed on Sep. 9, 2008.
(60) Provisional application No. 61/038,027, filed on Mar. 19, 2008.

(51) Int. Cl. A61K 47/36; A61K 9/127; A61K 47/28; A61K 31/65; A61K 47/14; A61K 47/06; G06F 19/00; A61K 31/704

(52) U.S. Cl. CPC A61K 47/36; G06F 19/3481; A61K 9/127; A61K 31/704; A61K 31/65; A61K 47/14; A61K 47/06; A61K 47/28
USPC 424/450; 514/34; 514/152; 424/489; 424/400

(57) ABSTRACT

Drug delivery compositions are disclosed that include multimolecular assemblies that have heparosan polymer(s) attached thereto and therapeutic(s) (and/or potential therapeutic(s)) entrapped, carried, or otherwise bound in the multimolecular assemblies. Methods for producing and using the drug delivery compositions are also disclosed.

### CROSS REFERENCE TO RELATED APPLICATIONS/INCORPORATION BY REFERENCE STATEMENT

The present application claims benefit under 35 USC §119(e) of U.S. Ser. No. 61/932,570, filed Jan. 28, 2014. The present application is also a continuation-in-part of U.S. Ser. No. 13/715,117, filed Dec. 14, 2014; which is a divisional of U.S. Ser. No. 12/556,324, filed Sep. 9, 2009. The '324 application claims benefit under 35 USC §119(e) of U.S. Ser. No. 61/179,275, filed May 18, 2009. In addition, the '324 application is also a continuation-in-part of U.S. Ser. No. 12/383,046, filed Mar. 19, 2009, now abandoned; which claims benefit under 35 USC §119(e) of U.S. Ser. No. 61/095,572, filed Sep. 9, 2008; and U.S. Ser. No. 61/038,027, filed Mar. 19, 2008. The entire contents of the above-referenced patent applications are hereby expressly incorporated herein by reference.

### STATEMENT REGARDING FEDERALLY SPONSORED RESEARCH OR DEVELOPMENT

Not Applicable.

### BACKGROUND

The delivery of many therapeutic molecules, including small molecular weight compounds (e.g., typically less than 10,000 Daltons) or drugs (including both hydrophilic and hydrophobic substances), is often problematic due to the therapeutic's physical, chemical, and/or biological properties. For example, the therapeutic may have a small size that results in the therapeutic being easily lost from the body, and/or the therapeutic may have aqueous solubility issues and/or issues with reactivity, toxicity, and/or off-target localization. Therefore, the entrapment of therapeutics into delivery systems such as liposomes, lipid vesicles, vesicles, micelles, or aggregates/assemblies/nanoparticles (here grouped as ‘lipid-based systems,’ ‘liposomes,’ or ‘micelles’ for the sake of simplicity) composed of fatty acids, lipid derivatives, sterols, and/or hydrophobic or amphiphilic molecules has shown some benefit, but such simple systems are not without defects. For example but not by way of limitation, naked lipid/fatty acid assemblies are often cleared very quickly in the mammalian body (e.g., on a time scale of minutes to hours).

One beneficial strategy to improve liposomes or micelles used for therapeutics involves masking/cloaking the surface of the liposome or micelle with a protective molecule to avoid clearing mechanisms (e.g., renal filtration or reticuloendothelial systems). One such protective molecule that has been employed is polyethylene glycol (PEG), a synthetic hydrophilic polymer with a hydrophobic anchor (e.g., an amphiphilic block co-polymer containing both a hydrophilic and a hydrydrophobic component). For example, an FDA-approved commercial product is DOXIL® (PEG-coated liposomes with entrapped doxorubicin; Janssen Products, LP; Titusville, N.J.) that allows the liposome to persist longer in the body while reducing the innate toxicity (in the case of doxorubicin, cardiotoxicity) of the trapped drug. While this strategy shows benefits in the short-term, after repeated dosing (for example, as needed with chemotherapy for cancer treatment), PEG-liposomes have exhibited unwanted side effects (e.g., rapid clearance by anti-PEG antibodies after multiple doses as well as hypersensitivity due to complement). Therefore, there is a need in the art for a more biocompatible protective molecule such as a ‘self’ molecule naturally found in the mammalian body that can be utilized instead of or in combination with, for example, PEG, as a protective molecule in liposome or micelle therapeutics.

PEGylation, a technology developed in the 1970-80s, is an FDA-approved process of adding PEG (the ‘vehicle’) to the therapeutic ‘cargo’ such as, for example, small molecule drugs, proteins, polynucleotides, micelles, and liposomes (Pasut et al., 2012; Veronese, 2001). PEG-drug conjugates show prolonged residence in the body, decreased degradation by metabolic enzymes, and reduced immunogenicity. However, while PEG, a synthetic polymer, is useful now, its artificial nature and chemical synthesis present increasingly serious drawbacks (Gaberc-Porekar et al., 2008; Armstrong et al., 2007; Ishida et al., 2007; Sundy et al., 2007; Hamad et al., 2008; and Verhoef et al., 2014). Notable concerns include PEG's degradation products (oxalic acid, aldehydes, and ketones), accumulation in some tissues (e.g., liver, kidney, brain), and inadequate upper size limit for controlled synthesis. Even more disturbing is the recently recognized increased incidence of PEG immunogenicity; the widespread use of PEG in consumer products has apparently sensitized many patients, so in certain cases, cargos that should be protected by PEG are actually cleared more quickly by anti-PEG. Due to the danger of potentially widespread failure, several companies are actively looking for PEG substitutes. Thus, development of a new approach to enhance drug performance has become urgent.

Two other biomaterials, poly[sialic acid] (PSA; Gregoriadis et al., 2000) and hydroxyethyl modified starch (HES; Besheer et al., 2009), have been proposed to be “PEG-substitutes.” PSA is polydisperse, will form aggregates unless modified, and sometimes triggers the immune system. HES is an approved plasma extender, but it has heterogeneous size and modification levels, breaks down in the blood (thus complicating pharmacokinetics), and can trigger corn allergies. PSA is not currently available in sizes >100 kDa, and HES >60 kDa accumulates in tissues.

Therefore, alternative modifying and/or coupling agents that can be used with drug delivery compositions, and which overcome the defects and disadvantages of the prior art, are continually being sought.

### BRIEF DESCRIPTION OF THE SEVERAL VIEWS OF THE DRAWINGS

FIG. 1A schematically depicts one strategy for adding a heparosan derivative with a hydrophobic group (HEP-HM) to a pre-formed drug-containing liposome. Chemical modification of heparosan ([−4-GlcNAc-α1,4-GlcUA-β1-]ₙ) with a hydrophobic species or anchor (black box) allows its incorporation into a liposomal delivery system containing various therapeutics (black circles; e.g., water soluble/polar small molecules, polypeptides, polynucleotides, drugs or drug candidates, etc). One or multiple HEP-HM chains can be added as desired. Here, a single reducing terminal position was chosen for the HM placement, but if desired, the HM may be located at the non-reducing terminus or at multiple positions along the backbone of HEP. The image on the left of the diagram depicts a HEPosome, and is a non-limiting example. Alternatively, instead of using pre-formed liposomes, the HEP-HM can also be added at the first or intermediate step(s) of liposomal preparation.

FIG. 1B schematically depicts one strategy for creating heparosan micelles loaded with drug or drug candidate. Chemical modification of heparosan ([−4-GlcNAc-α1,4-GlcUA-β1-]ₙ) with different hydrophobic species (HEP-HM, black triangle) allows it to form micelles that can act as a delivery system for relatively hydrophobic or non-polar drugs (white circle). The image on the left of the diagram depicts a HEPcelle, and is a non-limiting example. Alternatively, instead of using the drug-loaded micelle concept depicted here, the hydrophobic component on the HEP chain may be the drug or drug candidiate itself which serves as both the anchor/core of the micelle or nanoparticle; the linking bond can be either stable or labile (the latter to effect subsequent drug release). Here, a single reducing terminal position was chosen for the HM/drug placement, but if desired, the HM/drug may be located at the non-reducing terminus or at multiple positions along the backbone of HEP.

FIG. 2 depicts the schematic structures of a chemically modified heparosan derivative with a hydrophobic group (HEP-HM) employed for liposomal and/or micellar drug delivery compositions. Heparosan ([−4-GlcNAc-α1,4-GlcUA-β1-]ₙ) (HEP) can be linked with hydrophobic molecules (HM), including fatty acids and phospholipids (containing either a single or multiple acyl chains of various lengths as well as various saturation levels). In Panels A-C of FIG. 2, schematic representations of HEP conjugated to: (A) Palmitic acid N-hydroxysuccinimide ester; (B) 1,2-Dipalmitoyl-sn-Glycero-3-Phosphothioethanol; or (C)N-(Succinimidyloxy-glutaryl)-L-α-phosphatidylethanolamine, 1-Palmitoyl-2-oleoyl are shown. In addition, heparosan ([−4-GlcNAc-α1,4-GlcUA-β1-]ₙ) (HEP) can be linked with hydrophobic molecules (HM), including sterols, natural and/or synthetic lipids. Ine Panels D-F of FIG. 2, schematic representation of HEP conjugated to: (D) thiocholesterol; (E) 1,2-Distearoyl-sn-glycero-3-phosphoethanolamine; or (F)N-(heptadecalfluoroundecyl) maleimide are shown. Heparosan of any chain length can be used; for example, but not by way of limitation, the HEP polymer may be in a range of from about 800 Da to about 300 kDa.

FIG. 3 depicts an agarose gel demonstrating the synthesis of HEP-palmitoyl derivatives. Lanes: amine-containing heparosan 12 kDa (lane H); conjugate of amine-containing 26 kDa HEP reacted with palmitic acid N-hydroxysuccinimide ester (Sigma-Aldrich, St Louis, Mo. (lane R; the upper and lower arrows denote the product and starting reagent, respectively); purified 12-kDa or 26-kDa HEP-palmitoyl (two ‘C’ lanes); HA standards (lane L) (Select-HA LoLadder, Hyalose LLC, Oklahoma City, Okla.; molecular masses are indicated) were analyzed by 2% agarose gel and Stains-All (Sigma-Aldrich, St Louis, Mo.). The slower migration of the HEP-palmitoyl, in comparison to the starting material) in the gel indicates the formation of the claimed heparosan derivatives with hydrophobic groups.

FIG. 4 depicts a normal-phase silica TLC plate demonstrating the synthesis of a HEP-tetramer ([−4-GlcNAc-α1,4-GlcUA-β1-]ₙ where n=2) palmitoyl derivative. The starting heparosan tetramer (H), the HEP-tetramer palmitoyl conjugate (C), and the mixture of both (M) were analyzed using 2:1:1 n-butanol/acetic acid/H₂O as the running buffer and then stained with 0.2% naphthoresorcinol in ethanol staining solution followed by heating. The formation of the heparosan tetramer derivative with a hydrophobic group is indicated by the faster running product molecule (marked with asterisk).

FIG. 5A depicts an agarose gel demonstrating the synthesis of HEP-palmitoyl/oleoyl derivative. Heparosan 26 kDa (lane H) containing an amine group was treated with 1-palmitoyl-2-oleoyl N-hydroxysuccinimide ester (NOF America Corporation, White Plains, N.Y.). The reaction mixture (M) containing the HEP-amine starting material (bottom arrow) and HEP-palmitoyl/oleoyl product (top arrow), 26 kDa HEP (H), and HA standards (lane L) (Select-HA LoLadder, Hyalose LLC, Oklahoma City, Okla.; molecular masses are indicated) were analyzed with 1% agarose gel and Stains-All. The slow-running, smeared band observed for HEP-palmitoyl/oleyl indicates the formation of aggregates/micelles.

FIG. 5B depicts gel analyses of higher molecular weight heparosan derivatives (99, 168, or 307 kDa) treated with the same 1-palmitoyl-2-oleoyl N-hydroxysuccinimide ester as in FIG. 5A. The slow-running, smeared band (marked with arrows) observed for HEP-palmitoyl/oleyl indicates the formation of aggregates/micelles.

FIG. 6 depicts an agarose gel demonstrating the synthesis of HEP-palmitoyl derivative. Heparosan 29 kDa containing an amine group was treated with palmitic anhydride (Sigma-Aldrich, St Louis, Mo.). The reaction mixture (M) containing HEP starting material (bottom arrow) and HEP-palmitoyl product (top arrow), 29 kDa HEP-amine (H), and HA standards (lane L; Select-HA LoLadder) were analyzed with 1.5% agarose gel and Stains-All. HEP-palmitoyl was formed as indicated by the slower migration of the heparosan derivative with a hydrophobic group.

FIG. 7 depicts an agarose gel stained with Stains-All (Sigma-Aldrich, St Louis, Mo.) demonstrating the synthesis of HEP-dipalmitoyl derivatives. Heparosan 12 kDa or 42 kDa (lane H) carrying a reactive maleimide group was linked to the thiol-dipalmitoyl hydrophobic molecule (Avanti® Polar Lipids, Inc., Alabaster, Ala.); the resulting purified product (lane C) and HA standards (lane L; Select-HA LoLadder) were analyzed with 1% agarose gel and Stains-All. The agarose gel analysis demonstrated the self-association of most of the HEP-dipalmitoyl in micelles, but some portion runs apparently as monomers.

FIG. 8 shows the size exclusion chromatography analyses of hydrophobic HEP-dipalmitoyl derivatives. The 12 kDa or 40 kDa HEP-dipalmitoyl derivatives were separated on SEPHAROSE® 6B resin (GE Healthcare Bio-Sciences, Pittsburgh, Pa.) in Tris-buffered saline and the different fractions were analyzed by the carbazole assay for glucuronic acid content (panel A), and by 6% PAGE gel with Stains-All detection (panel B). The presence heparosan in the void volume of the column and the analysis of the corresponding fractions (F#) with slow running bands on the gel indicates that the HEP-dipalmitoyl derivative forms assemblies composed of many molecules, namely micelles or aggregates.

FIG. 9 shows the size exclusion chromatography purification of HEP (no HM) in comparison the hydrophobic HEP-monopalmitoyl. As performed in FIG. 8, the different fractions of HEP 38 kDa (with no HM) or 25 kDa HEP-palmitoyl were analyzed by carbazole assay for glucuronic acid content (panel A), and analyzed by 6% PAGE gel (panel B). Under these conditions, the two samples migrated as predicted for their monomeric sizes on both the column and the gel, and thus did not form micelles or aggregates under these conditions.

FIG. 10 depicts an agarose gel stained demonstrating the synthesis of a HEP-cholesterol derivative. Heparosan 12.5 kDa carrying a maleimide group was linked to thiolcholesterol hydrophobic molecule (Sigma-Aldrich, St Louis, Mo.). The 1.5% agarose gel shows HA standards (L; Select-HA LoLadder), HEP-maleimide 12.5 kDa (H), and the reaction mixture (M) containing the HEP-thiocholesterol conjugate (upper arrow) and the unreacted HEP (bottom arrow; note this particular reaction did not go to completion thus unmodified HEP remains). The agarose gel analysis demonstrated the association of most of the HEP-thiocholesterol in micelles or aggregates as indicated by the slowly migrating smeared distribution of the product.

FIG. 11 depicts an agarose gel demonstrating the synthesis of HEP polymers conjugated to distearoyl phosphatidylethanolamine (Sigma-Aldrich, St Louis, Mo.). Heparosan 26 kDa carrying a squarate group were linked to distearoyl phosphatidyethanolamine. The 1.5% agarose gel shows HA standards (L; Select-HA LoLadder), HEP-squarate 26 kDa (H), and the reaction mixture (M) containing the HEP-distearoyl (upper arrow) and the unreacted HEP (bottom arrow). The agarose gel analysis demonstrated the association of much of the HEP-distearoyl in micelles or aggregates as indicated by the slowly migrating smeared distribution of the product.

FIG. 12 depicts an agarose gel demonstrating the synthesis of a HEP-HM by reacting a 29 kDa sulfhydryl-containing HEP polymer derivative with N-(4,4,5,5,6,6,7,7,8,8,9,9,10,10,11,11,11-heptadecafluoroundecyl)maleimide (HDFU-maleimide; Sigma-Aldrich, St Louis, Mo.). The 1% agarose gel shows HA standards (L; Select-HA LoLadder), starting HEP-polymer alone (H), and reaction mixture (M) containing the HEP-HDFU derivative (upper arrow) and the unreacted HEP (bottom arrow). The agarose gel analysis demonstrated the self-association of much of the HEP-HDFU as indicated by the slowly migrating smeared distribution of the product.

FIG. 13 shows an agarose gel analysis of the incorporation of HEP-palmitoyl into liposomes. On the left panel, a 2% agarose gel was loaded with HA standards (lane L; Select-HA LoLadder), a semi-purified mixture of a 28 kDa HEP-palmitoyl synthesis reaction (lane H) [HEP-palmitoyl product (upper arrow) and free HEP amine starting material (lower arrow)], or liposome only (lane Lp; preparation #3, preloaded with carboxyfluorescein dye) and stained with Stains-All. On the 2% gel in the right panel, the samples were the semi-purified preparation (lane H), this same HEP-palm/HEP mixture plus liposomes (lane R; no separation by centrifugation), or the resulting centrifuged pellets containing the liposomal fraction from such liposome+HEP-HP admixtures after 7 days, 1 day, or 15 minutes of incubation (lanes marked ‘7d,’ ‘1d,’ or ‘15 min,’ respectively). These liposomes alone did not stain. The HEP without the HM did not pellet with the liposomes. The HEP-palmitoyl was incorporated into the liposomes as indicated by the smeared, slower mobility bands (marked with asterisk) in the lanes of pellets from the time course.

FIG. 14 depicts an agarose gel analysis of the interaction of purified HEP-palmitoyl derivative and liposomes. A titration of 28 kDa HEP-palmitoyl (HP; black triangle indicates the relative concentrations employed) with a fixed amount of liposomes (prep #3) was performed. After incubation, centrifugation was used to separate the free HP from the liposomal-bound HP and the presence of the sugar then was detected by gel/Stains-All analysis. The gel was loaded so that equal mass amounts of HP (based on the initial reaction) were loaded on the gel (i.e., less volume of the more concentrated HP reaction samples was loaded per lane and vice versa). The upper panel lanes are the pellets (liposome-bound HP) while the lower panel lanes are the supernatants (free HP). The ‘HP+liposomes’ (on right) shows the presence of heparosan in pellet with liposomes; conversely, the negative controls without liposomes (′HP′ on the left) shows the staining in the supernatants, but not the pellets. A saturation effect is seen; at low concentrations, most HP is with the liposome, but at higher levels there is excess HP and is seen more in the supernatants because the liposomes only have so much HP-binding capacity due to a finite surface area (due to the normalization of equal input HP loading per lane there is not ever-increasing amounts of HP in the pellet in an effort to avoid over-loading of the gel for better quantification) (S=HEP-palmitoyl standard; 1 or 2 micrograms, top and bottom panels, respectively).

FIG. 15 depicts an agarose gel analysis of 12 kDa HEP-palmitoyl incorporation into non-PEGylated DOXOSOME® (doxorubicin liposomes; Encapsula Nanosciences LLC, Brentwood, Tenn.). The liposomes with entrapped drug (prep #5) were mixed with HEP-palmitoyl to test the effect of incubation temperature and the HEP-HM concentration (either 0.5 or 1 mol % final based on lipid content). After 2 hours of incubation in phosphate buffered saline buffer, the samples were centrifuged and the pellets with liposomes were separated on a 2% agarose gel with Stains-All detection (HA standards lane L; Select-HA LoLadder). The liposomes incorporated significant amounts of this HEP-HM and did not leak appreciable amounts of drug.

FIG. 16 depicts an agarose gel analysis of HEP-lipid drug-loaded liposomes (FIG. 15). Either a solution of 28 kDa HEP-palmitoyl (P) or 13 kDa HEP-dipalmitoyl (P2) or water alone (a negative control) (0) were mixed with drug-loaded liposomes (prep #4 with either entrapped doxorubicin ‘D’ or tetracycline ‘T’ as noted; drug-saturated ethanol solution was mixed with lipids in ethanol, then extruded into 10 volumes of vigorously mixed saline). After a 30-minute incubation, the heparosan/liposome mixtures were diluted with saline buffer, and centrifugation was used to separate the free HEP-lipid from the bound HEP-lipid in liposomes. Samples run on the gel were either the starting mixture (′start′ which reflects the total input materials) or the washed pellet (‘p’ which reflects the liposomally-associated materials). The presence of the sugar was detected by gel/Stains-All analysis. The presence of the drugs was detected by visible color/ultraviolet induced fluorescence (red/red for D and yellow/green for T). The HEP-HM and drug fractionated with the liposomes in the pellets as seen by the bands in the ‘p’ lanes in the two sets of lanes on the right half of the gel.

FIG. 17 is a plot that illustrates the protection of HEPosomes from the effects of a pore-forming toxin. Liposomes (prep #3 with entrapped carboxyfluorescein dye) were either (i) untreated (‘control’), or (ii) incubated with various amounts of HEP-palmitoyl derivative (HP) or (iii) with various amounts of HEP alone (H; no hydrophobic moiety). Then toxin was added (about 3× the concentration needed for complete lysis of liposomes) and incubated for 1 hour at 37° C. Liposome destruction by the toxin was followed by assessing the increase in dye release from the liposomes via loss of fluorescence quenching. The line graph depicts a titration of 28 kDa HEP-palmitoyl (HP; grey circles) or 28 kDa HEP (H; lacking a hydrophobic moiety; grey squares), and water (control; black triangles). Only HEP-palmitoyl was found to protect the liposome against the pore forming toxin (H or water samples were completely lysed by toxin) indicating that the surface of the liposome was coated with HEP chains from the HEP-palmitoyl derivative.

FIG. 18 depicts the efficacy of HEP-coated (1 molar %) Doxorubicin-entrapped liposomes (a type of HEPosome) to inhibit tumors, as determined using the 4T1 murine mammary tumor model. The 4T1 tumor-bearing Balb/c mice received HEP-coated DOXOSOME®s (HEP; black squares), PEGylated DOXOSOME®s (PEG; black upward pointing triangles), naked (only lipids and cholesterol; black downward pointing triangles) DOXOSOME®s at 6 mg/kg, or vehicle only (black circles) at day 10 and 20 post implantation (marked as ‘Tx’). Tumors were measured by caliper to give a tumor volume. Statistical significance (*) was determined by ANOVA. The efficacy study indicates a comparable response between the three DOXOSOME® formulations with improved efficacy arising in the HEP-coated DOXOSOME® treated group compared to the naked group.

FIG. 19 depicts a chromatographic plot illustrating the drug carrying capacity of HEPcelles. Size exclusion chromatography analyses (SEPHAROSE® 6B; GE Healthcare Bio-Sciences, Pittsburgh, Pa.) of heparosan (without a HM) or two HEP-HM derivatives were used to assess if primuline (P), a non-polar cargo, can be carried by HEP-containing micelles. Various polymers (150 μg of 38 kDa HEP, 25 kDa HEP-palmitoyl, or 40 kDa HEP-dipalmitoyl) were incubated with 2.5 μg primuline at room temperature under shaking conditions for 20 minutes. The fluorescence of the fractions was monitored to assay for the presence of primuline in a hydrophobic environment (this molecule does not have a high quantum efficiency in a hydrophilic or aqueous environment). The fluorescence peak was observed only in the void volume of the HEP-dipalmitoyl sample demonstrating that hydrophobic primuline molecules were incorporated into micelles or nanoparticle aggregates with a hydrophobic core.

### DETAILED DESCRIPTION

Before explaining at least one embodiment of the presently disclosed and/or claimed concept(s) in detail, it is to be understood that the presently disclosed and/or claimed concept(s) is not limited in its application to the details of construction and the arrangement of the components or steps or methodologies set forth in the following description or illustrated in the drawings. The presently disclosed and/or claimed concept(s) is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the presently disclosed and/or claimed concept(s) shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those of ordinary skill in the art to which this presently disclosed and/or claimed concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions/complexes, kits, and/or methods disclosed or otherwise contemplated herein can be made and executed without undue experimentation in light of the present disclosure. While the compositions/complexes, kits, and methods of the presently disclosed and/or claimed inventive concept(s) have been described in terms of particular embodiments, it will be apparent to those of ordinary skill in the art that variations may be applied to the compositions/complexes, kits, and/or methods as well as in the steps and/or in the sequence of steps of the methods described herein without departing from the concept, spirit, and scope of the presently disclosed and/or claimed inventive concept(s). All such similar substitutes and modifications apparent to those of ordinary skill in the art are deemed to be within the spirit, scope, and concept of the presently disclosed and/or claimed inventive concept(s).

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, and/or CAB. Continuing with this example, expressly included are combinations that contain repeats of one or more item or term, such as BB, AAA, AAB, BBC, AAABCCCC, CBBAAA, CABABB, and so forth. One of ordinary skill in the art will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “heparosan” as used herein will be understood to refer to a carbohydrate chain with a repeat structure of ([−4-N-acetylglucosamine-α1,4-glucuronic acid-β1-]ₙ), wherein n is 1 or greater. In certain non-limiting examples, n may be from about 2 to about 5,000. The term “oligosaccharide” generally denotes n being from about 1 to about 11, while the term “polysaccharide” denotes n being equal to or greater than 12. The term “heparosan” may be utilized interchangeably with the terms “N-acetylheparosan” and “unsulfated, unepimerized heparin.”

The term “hydrophobic molecule” (HM) as used herein refers to moieties with relatively non-polar and/or hydrocarbon-rich and/or perfluorinated structures. Typically, these structures will partition into hydrophobic regions or environments or multi-molecular assemblies, including acyl chains of lipid bilayers or the hydrophobic cores of micelles due to the “hydrophobic interaction” and/or van der Waals forces.

The term “HEPosome” as used herein refers to heparosan-containing or heparosan-coated liposomal assemblies or nanoparticles with an aqueous lumen region that carries therapeutic agents/potential therapeutic agents. In addition, in certain embodiments, therapeutic/potential therapeutic agents may be carried in the hydrophobic portion of the lipid components.

The term “HEPcelle” as used herein refers to heparosan-containing or heparosan-coated micellar assemblies or aggregates or nanoparticles with a hydrophobic core region (e.g., lipid acyl tails or alkyl/aryl groups or fluorine-containing moieties) that carries therapeutic agents/potential therapeutic agents.

The term “UDP-sugar” as used herein refers to a carbohydrate precursor modified with uridine diphosphate (e.g., UDP-N-acetylglucosamine).

The term “polypeptide” as used herein will be understood to refer to a polymer of amino acids. The polymer may include d-, I-, and/or artificial variants of amino acids. In addition, the term “polypeptide” will be understood to include peptides, proteins, and glycoproteins.

The term “polynucleotide” as used herein will be understood to refer to a polymer of nucleotides. Nucleotides, as used herein, will be understood to include deoxyribose nucleotides and/or ribose nucleotides, as well as artificial variants thereof.

The term “analog” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all; in the latter case, these drugs can often act as bait or blockers of activity. Alternatively and/or in addition thereto, for a chemical, an analog may be any structure that has the functionalities (including alterations or substitutions in the core moiety) desired, even if comprised of different atoms or isomeric arrangements.

The term “conjugate” as used herein refers to a complex created between two or more compounds by covalent or weak bonds. The term “covalent” as used herein refers to the sharing of electrons between atoms to create a chemical interaction.

The term “cargo” as used herein refers to the therapeutic agent (i.e., drug), potential therapeutic agent (i.e., drug candidate), or other biologically active component in the liposome or micelle, while the term “delivery system” as used herein refers to the carrier of the cargo (e.g., the heparosan-coated liposome or micelle).

The term “amphiphile” as used herein is defined as a molecule comprised of both a relatively hydrophilic part (e.g., polar, water-loving) and a relatively hydrophobic (e.g., nonpolar, water-hating) part; these types of molecules are described as “amphiphilic.”

As used herein, the terms “active agent(s),” “active ingredient(s),” “pharmaceutical ingredient(s),” “therapeutic,” “medicant,” “medicine,” “biologically active compound” and “bioactive agent(s)” are defined as drugs and/or pharmaceutically active ingredients.

The term “Dalton” (Da) as used herein will be understood to refer to a unit of molecular mass for polypeptides and polysaccharides. The term “kiloDalton” (kDa) as used herein refers to one thousand Daltons. The term “megaDalton” (MDa) as used herein will be understood to refer to one million Daltons (i.e., one thousand kDa).

The term “polydispersity” as used herein refers to a measure of the width of molecular weight distributions of a product. In one, non-limiting example, polydispersity is calculated by dividing the Weight average molar mass (Mw) by the Number average molar mass (Mn); thus, polydispersity=Mw/Mn.

The terms “quasi-monodisperse” and “substantially monodisperse” are used herein interchangeably and will be understood to refer to very narrow size distributions approaching the ideal polydispersity value of 1.

The term “PEGylation” as used herein refers to the modification of a drug or drug candidate molecule by addition of polyethylene glycol thereto.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism. A molecule/composition can be biologically active through its own functionalities, or may be biologically active based on its ability to activate, modulate, or inhibit molecules/compositions having their own biological activity. In addition, biological activity observed in in vitro proxy models is indicative of in vivo action of a molecule/composition.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “patient” as used herein includes human and veterinary subjects. “Mammal” for purposes of treatment refers to any animal classified as a mammal, including human, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition or disorder as well as individuals who are at risk of acquiring a particular condition or disorder (e.g., those needing prophylactic/preventative measures). The term “treating” refers to administering an agent to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, or management of a disease and/or condition. The specific amount that is therapeutically effective can be readily determined by the ordinary medical practitioner, and can vary depending on factors known in the art, such as the type of disease/cancer, the patient's history and age, the stage of disease/cancer, and the co-administration of other agents.

A “disorder” is any condition that would benefit from treatment with the compositions disclosed herein. This includes chronic and acute disorders or diseases including those pathological conditions which predispose the mammal to the disorder in question.

The term “effective amount” refers to an amount of a biologically active molecule or complex or derivative thereof sufficient to exhibit a detectable therapeutic effect without undue adverse side effects (such as toxicity, irritation and allergic response) commensurate with a reasonable benefit/risk ratio when used in the manner of the inventive concept(s). The therapeutic effect may include, for example but not by way of limitation, inhibiting the growth of microbes and/or opportunistic infections. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The terms “administration” and “administering,” as used herein will be understood to include all routes of administration known in the art, including but not limited to, oral, topical, transdermal, parenteral, subcutaneous, intranasal, mucosal, intramuscular, intraperitoneal, intravitreal, and intravenous routes, including both local and systemic applications. In addition, the compositions of the presently disclosed and/or claimed inventive concept(s) (and/or the methods of administration of same) may be designed to provide delayed, controlled or sustained release using formulation techniques which are well known in the art.

The following abbreviations, which may be utilized herein, will be understood to refer the following terms or phrases: Strong anion exchange chromatography [SAX]; PolyAcrylamide Gel Electrophoresis [PAGE]; dimethylsulfoxide [DMSO]; Poly(ethylene Glycol) [PEG]; Hydroxyethylstarch [HES]; Poly(sialic acid) [PSA]; Uridine diphosphate [UDP]; Heparan Sulfate [HS]; Glycosaminoglycan [GAG]; Hydrophobic Molecule [HM]; Dalton [Da]; Kilodalton [kDa]; Molecular Weight [MW]; Molar [M]; Gram [g]; Kilogram [kg]; Milligram [mg]; Microgram [μg or ug]; Nanogram [ng]; Volt [V]; High Performance Liquid Chromatography-Size Exclusion Chromatography [HPLC-SEC].

Turning now to the presently disclosed and/or claimed inventive concept(s), a new drug delivery platform has been developed that overcomes the defects and disadvantages of the prior art discussed herein above. This new drug delivery platform may be utilized alone or in combination with another half-life extension technology, such as but not limited to, the PEGylation method. Heparosan ([−4-GlcNAc-α1,4-GlcUA-β1-]ₙ) (HEP) is a natural polysaccharide related to heparin (it is the unsulfated, unepimerized backbone of heparin and heparan sulfate [HS]), one of the most widely used drugs in pharmacopeias. Heparosan should be biocompatible in the human body because it is a natural precursor in the heparin/HS biosynthetic pathway, and stretches of heparosan exist in human heparan sulfate chains (e.g., approximately 30% of the disaccharide units in human liver HS). Certain pathogenic bacteria even exploit the “self” nature of heparosan by using a heparosan coating to evade the immune system during infection (DeAngelis, 2002). The presently disclosed and/or claimed drug delivery plaftform that includes attachment of heparosan a multimolecular assembly vehicle (e.g., liposomes, micelles, etc.) has many potentially superior attributes over analogous platforms modified by PEGylation. These attributes include, but are not limited to: a) ease of generating a larger size range of polymers, b) higher water solubility, c) greater biocompatibility of degradation products, and d) no accumulation (or only an immaterial degree of accumulation) in tissues over the long-term. For example, if heparosan is internalized into cells (e.g., via pinocytosis, fluid phase uptake, etc.) and transported to lysosomes, it should be degraded by resident glucuronidase and hexosaminidase enzymes, similar to heparin or hyaluronan; the data in rats and primates presented herein verified this prediction. A key advantage for therapeutic modifications with heparosan is that its degradation products, GlcNAc and GlcUA, are normal monosaccharides in animals and thus are non-toxic (unlike PEG metabolites) and can be recycled by cells.

In a 2008 Current Opinions in Drug Discovery and Development article (Gaberc-Porekar et al., 2008), it was predicted that future drugs will use higher molecular weight PEGs and/or be given at higher doses for long periods. Because of PEG's intrinsic limitations and emerging immunogenicity, the multimolecular assemblies containing heparosan is a preferable therapeutic vehicle.

The PEG vehicle, although a very useful polymer, has growing liabilities. The artificial nature of PEG is a concern for pharmaceuticals used at high doses and/or for long duration treatments, because the liver detoxification system can create a variety of reactive PEG metabolites that are cytotoxic. In contrast, the presently disclosed and/or claimed drug delivery compositions utilize natural heparosan polymers linked to a hydrophobic molecule that anchors the polymer to a multimolecular assembly (i.e., liposome, micelle, or other lipid aggregate); the heparosan is degraded into normal sugars and recycled, and thus should possess substantially lower toxicity when compared to PEG.

Another biocompatibility issue is that PEG, present in certain drug formulations, can induce anti-PEG antibodies in some patients. Anti-PEG antibodies may also be triggered by widespread use of PEG in many ‘consumer’ products (e.g., toothpaste, laxatives, vitamin pills, antacids, etc.). In 1984, it was reported that in naïve persons, anti-PEG antibodies were detected in approximately 0.2% of the samples, but as of 2001, stunningly, approximately 22 to 25% of healthy blood donor (n=350) samples had anti-PEG IgM or IgG. Obviously, anti-PEG antibodies can limit the usefulness of PEG therapeutics. For example, some leukemia patients no longer respond to PEG-asparaginase (ONCASPAR®, Enzon Inc., Bridgewater, N.J.) medication due to anti-PEG antibody levels. Similarly, anti-PEG was found in gout patients treated with PEG-uricase (KRYSTEXXA®, Crealta Pharmaceuticals LLC, Lake Forest, Ill.); these persons were refractory to therapy as the drug was cleared rapidly. Also, it was reported in the case of the PEG-doxorubicin liposomes, DOXIL® (Janssen Products, LP; Titusville, N.J.) that rapid clearance and hypersensitivity was observed due to anti-PEG antibodies.

Some patients react immunologically to PEG after exposure to certain formulations especially liposomes. Since heparosan is a naturally occurring sugar polymer in mammals, it should not be immunogenic. Generally speaking, molecules that normally exist in the body are regarded as “self” and therefore not subjected to attack by antibodies, phagocytes, or the complement system. This fact is employed by certain pathogenic bacteria that camouflage themselves with heparosan molecules; Pasteurella multocida Type D and Escherichia coli K5 both produce heparosan coatings that hide them from many host defenses. Only a few monoclonal antibodies to the heparosan polymer have ever been reported; the rare anti-heparosan producing clones were identified by extensive screening after immunizing with extremely antigenic bacterial membranes or key-hole limpet hemocyanin conjugates.

In addition, PEG can activate the complement system (via pattern receptors), which may explain why PEG triggers anaphylactic shock and/or other allergic reactions in some patients. Heparosan polymer, as a naturally occurring ‘self’ polymer in humans, was well tolerated in a recent rat study (approximately 100 mg/kg dosing; see, for example, US Application No. 2010/0036001 and U.S. Ser. No. 14/536,003; the entire contents of each of which are expressly incorporated herein by reference). Therefore, HEP-based therapeutics offer new options for treatment and hope to patients who cannot use PEG-based compounds, and in time these HEP-based therapeutics may replace PEG-based compounds.

Without O-sulfation on the polymer chain, heparosan is resistant to cleavage by the heparanase that typically digests heparin. Also, heparosan is not bound by HARE, a liver receptor that normally clears hyaluronan and heparin very efficiently from the bloodstream. Thus, the inventors hypothesized that heparosan would be very stable and would persist in the extracellular spaces of the body. A pharmacokinetics study in rats and monkeys showed that indeed heparosan had a long plasma half-life (t_{1/2} ranging from approximately 48-192 hours, depending on polymer size and animal species, in a 2-compartment model; see, for example, US Application No. 2010/0036001 and U.S. Ser. No. 14/536,003, incorporated supra).

If heparosan is internalized into cells (e.g., via pinocytosis, etc.) and transported to lysosomes, it should be degraded by resident glucuronidase and hexosaminidase enzymes, similar to heparin or hyaluronan; the data in rats verified this prediction (see, for example, US Application No. 2010/0036001 and U.S. Ser. No. 14/536,003, incorporated supra). A key advantage for therapeutic modifications with heparosan is that its degradation products, GlcNAc and GlcUA, are normal monosaccharides that are non-toxic (unlike PEG metabolites) and that can be recycled by cells.

The normal roles of heparin/heparan sulfate in vertebrates include inhibiting blood clotting. It was verified that without O-sulfation on the polymer, heparosan does not affect clotting of human plasma even when used at 15,000-fold higher levels than heparin on a mass basis (no difference in values between heparosan and saline control in aPTT and anti-Factor Xa diagnostic assays). The effect of high doses of heparosan (100 mg per kg body weight) was tested in healthy rats via intravenous injection (once on Day 1 and 8); this translates to at least approximately 100 to 2,000-fold higher levels than would be expected for use in humans. On Day 10, there were no adverse effects, as measured by blood or urine chemistry, hematology, or histology (see, for example, US Application No. 2010/0036001 and U.S. Ser. No. 14/536,003, incorporated supra).

The inventor has cloned, sequenced, and published a bacterial polymerizing enzyme called heparosan synthase, PmHS1. In 2007, this catalyst was harnessed for the chemoenzymatic synthesis of sugar polymers with a very narrow size distribution; these sugar polymers were characterized as being ‘monodisperse.’ The use of an acceptor in PmHS1-catalyzed reactions synchronizes polymerization, and the concentration of acceptor tightly controls the size of the heparosan product. Depending on the size, the polydispersity ranges from 1.06-1.18 (1=ideal); see U.S. Pat. No. 8,088,604, issued to DeAngelis et al. on Jan. 3, 2012, the entire contents of which are expressly incorporated herein by reference. This patented method is also amenable to preparing defined and reproducibly chemically activated heparosan to facilitate coupling to amine, sulfhydryl, aldehyde, or other reactive groups of the drug delivery platform components (e.g., a liposome, micelle, or the hydrophobic molecule anchor).

Two other biomaterials, poly[sialic acid] (PSA; Gregoriadis et al., 2000) and hydroxyethyl modified starch (HES; Besheer et al., 2009), have been proposed to be “PEG-substitutes.” PSA is polydisperse, will form aggregates unless modified, and sometimes triggers the immune system. HES is an approved plasma extender, but it has heterogeneous size and modification levels, breaks down in the blood (thus complicating pharmacokinetics), and can trigger corn allergies. PSA is not currently available in sizes >100 kDa. HES greater than 60 kDa accumulates in tissues. However, 800 Da to 4,500 kDa heparosan can be manufactured, thus providing the potential for longer half-life potential.

Alternative modifying and/or coupling agents that can be used with drug delivery compositions, and which overcome the defects and disadvantages of the prior art, are continually being sought. In summary, heparosan is a stealthy molecule well suited for use as a drug delivery vehicle component. Furthermore, molecularly defined heparosan polymers should facilitate quality control and FDA approval.

TABLE 1
Characteristics of Drug Delivery Compositions
The various polymers have different production methods, purity,
and biological behavior in the body. The ideal delivery agent should
have a “Yes” response in all critical categories below.

| | Quasi- | | |
|---|---|---|---|
| Polymer | Monodisperse? | Biodegradable? | Not Immunogenic? |
| PEG | Yes | No | Sometimes |
| Heparosan | Yes | Yes | Yes |
| HES | No | Yes | Yes |
| PSA | No | Yes | Sometimes |

(PEG = poly(ethylene glycol); HES = hydroxyethylstarch; PSA = poly(sialic acid); quasi-monodisperse [i.e., very narrow size distributions approaching the ideal polydispersity value of 1])

A liposome is an artificially prepared vesicle composed of a lipid bilayer. The major types of liposomes are the multilamellar vesicle (MLV), the small unilamellar vesicle (SUV), the large unilamellar vesicle (LUV), and the cochleate vesicle. These various lipid aggregates may be made by physical or mechanical methods (e.g., hydrating, homogenizing, or sonicating films), solvent replacement (e.g., organic to aqueous transition via evaporation, injection, or extrusion), or detergent removal (Wagner et al., 2011). The specific production method for liposomes is not key for the presently disclosed and/or claimed inventive concept(s), but rather the presence and use of a heparosan derivative or anchor in such liposomes used as drug delivery platforms.

A micelle is an aggregate of surfactant or lipid or amphiphilic molecules dispersed in a liquid colloid. A typical micelle in aqueous solution forms an aggregate with the hydrophilic regions in contact with surrounding solvent, sequestering the hydrophobic or non-polar regions in the micelle center. Micelles are approximately spherical in shape. Other phases, including shapes such as ellipsoids, cylinders, and bilayers, are also possible. In addition, polymeric micelles prepared from certain amphiphilic co-polymers consisting of both hydrophilic and hydrophobic monomer units can be used to carry drugs that have poor aqueous solubility.

In another embodiment, if the therapeutic/potential therapeutic agent itself is sufficiently hydrophobic or non-polar, and if the hydrophilic chain (e.g, PEG, heparosan, etc.) could be directly attached to the therapeutic/potential therapeutic agent, thereby forming an amphiliphilic structure, this conjugate itself would self-assemble into micellar or nanoparticulate structures without the need for other components. Depending on the therapeutic/potential therapeutic agent's requirements for activity in the body and/or interaction with its target, the linking bond between the two components may need to be cleavable or labile (e.g., when the conjugate enters the correct environment with an acidic pH such as in the lysosomes, etc., or by a metabolism via the patient's enzymes like protease or lipase, etc.) for therapeutic action.

Methods akin to the liposomal preparation may be employed for micellar production, but often the micelles can self-assemble in aqueous solution. The specific production method for micelles is not key for the presently disclosed and/or claimed inventive concept(s), but rather the presence and use of heparosan derivative or anchor in such micelles used as drug delivery platforms.

Liposomes are a common vehicle currently used for drug or therapeutic delivery. They can store their cargo or payload in their hydrophobic shell or their hydrophilic interior, depending on the nature of the agent being carried. A liposome encapsulates a region of aqueous solution inside a hydrophobic membrane; most dissolved hydrophilic solutes cannot readily pass through the lipid layer. Hydrophobic chemicals can be dissolved into the membrane, and in this way liposome can carry both hydrophobic molecules and hydrophilic molecules. To deliver the molecules to sites of action, the lipid bilayer can fuse with other bilayers such as the cell membrane, thus delivering the liposome contents. By making liposomes in a solution of drugs or therapeutics which would normally be unable to diffuse through the membrane, the liposome can be delivered past the cell's bilayer. Alternatively, under some conditions, the liposome will unassemble, thereby releasing the cargo.

Some problems with using liposomes in vivo are: (a) their immediate or relatively rapid uptake and clearance by the reticuloendothelial system (RES) or mononuclear phagocyte system (MPS) system (RES is an older term for the MPS), and (b) their relatively low stability in vitro. The RES/MPS is the part of the immune system that consists of the phagocytic cells located in reticular connective tissue. The cells are primarily monocytes and macrophages that accumulate in lymph nodes and the spleen. The Kupffer cells of the liver and tissue histiocytes are also part of the RES/MPS.

To combat clearance, molecules such as block copolymers, lipid molecules, and/or hydrophobic anchors containing the polyethylene glycol (PEG) polymer can be added to the surface of the liposomes or micelles. Increasing the mole percent of the PEG-containing component on the surface of the liposomes by 4-10% significantly increased circulation time in vivo from 200 to 1000 minutes. These liposomes are known as “stealth liposomes,” and are constructed with PEG studding the outside of the membrane. The PEG coating, which is somewhat inert in the body, allows for longer circulatory life for the drug delivery mechanism. The presently disclosed and/or claimed inventive concept(s) involves the use of another polymer, heparosan, which lacks some of the liabilities of PEG, as discussed in detail herein above. In addition to a PEG coating, some stealth liposomes also have some sort of species attached as a ligand on the liposome in order to enable binding via a specific expression on the targeted drug delivery site. These targeting ligands could be monoclonal antibodies (making an immunoliposome), vitamins, or specific antigens. Targeted liposomes can target nearly any cell type in the body and deliver drugs that would naturally be systemically delivered. Naturally toxic drugs can be much less toxic if delivered only to diseased tissues. In case of tumor treatment, certain anticancer drugs such as doxorubicin (DOXIL®, PEG-coated liposomes with entrapped doxorubicin; Janssen Products, LP; Titusville, N.J.), daunorubicin, and cisplatin are provided through liposomes. For infectious disease, therapeutics such as (but not limited to) antibiotics can be delivered. For inflammation, therapeutics such as (but not limited to) steroids and inhibitors can be delivered. For deficiencies, therapeutics such as (but not limited to) nutrients and vitamins or missing metabolites/factors can be delivered.

Certain embodiments of the presently disclosed and/or claimed inventive concept(s) relate in general to the field of the therapeutic drug cargo, which include lipid vesicle, liposome, micelles, and similar assemblies decorated with heparosan (HEP) ([−4-N-acetylglucosamine-α1,4-glucuronic acid-β1-]ₙ) polymer linked to a hydrophobic molecule (HM).

One non-limiting embodiment of the presently disclosed and/or claimed inventive concept(s) is directed to a drug delivery composition that includes a multimolecular assembly formed of a plurality of components, wherein at least a portion of the plurality of components includes at least one hydrophobic moiety, and wherein the plurality of components aggregate together via hydrophobic interactions to form the multimolecular assembly. The drug delivery composition also includes at least one heparosan polymer having a hydrophobic moiety-containing component attached thereto, wherein the hydrophobic moiety is incorporated into the multimolecular assembly. In this manner, the at least one heparosan polymer is attached to a surface of the multimolecular assembly.

In addition, the drug delivery composition also includes at least one therapeutic agent or potential therapeutic agent entrapped, carried, and/or bound in the multimolecular assembly. The therapeutic/potential therapeutic agent may be any agent capable of being entrapped, carried, and/or bound in the multimolecular assembly and functioning in accordance with the presently disclosed and/or claimed inventive concept(s). Particular examples of substances that may be utilized as the therapeutic/potential therapeutic agent include, but are not limited to, a chemotherapy agent, an antineoplastic agent, a steroid, an antibiotic, an anti-inflammatory agent, an agent that has an action on a central nervous system of the mammalian patient, an antihistaminic, an antiallergic agent, an antipyretic, a respiratory agent, an antimicrobial agent, an antihypertensive agent, a calcium antagonist, an antipsychotic, an agent for Parkinson's disease, a vitamin, an antitumor agent, a cholinergic agonist, a mydriatic, an antidepressant agent, an antidiabetic drug, an anorectic agent, an antimalarial agent, a polypeptide therapeutic, a cytokine, a hormone, an enzyme, an antibody, an antibody fragment, an antiulcerative agent, an anticancer agent, a vaccine antigen, a polynucleotide, a nutrient, a small molecule, and combinations thereof.

Any type of multimolecular assembly known in the art or otherwise contemplated herein that is capable of entrapping, carrying, and/or binding to a therapeutic/potential therapeutic agent and that is capable of having heparosan polymer(s) on a surface thereof may be utilized in accordance with the presently disclosed and/or claimed inventive concept(s). For example, but not by way of limitation, the multimolecular assembly may be a monolayer, such as but not limited to, a micelle. Particular, non-limiting examples of micelles that may be utilized in accordance with the presently disclosed and/or claimed inventive concept(s) include micelles that have a spherical, ellipsoid, and/or cylindrical shape and/or polymeric micelles. Alternatively, the multimolecular assembly may be a bilayer, such as but not limited to, a vesicle and/or liposome. Particular, non-limiting examples of vesicles that may be utilized in accordance with the presently disclosed and/or claimed inventive concept(s) include a multilamellar vesicle (MLV), a small unilamellar vesicle (SUV), a large unilamellar vesicle (LUV), a cochleate vesicle, and combinations thereof. In yet another alternative, the multimolecular assembly may be a combination of any of the above, such as but not limited to, an aggregate of micelle(s) and/or vesicle(s).

Any type of component known in the art or otherwise contemplated herein that includes at least one hydrophobic moiety and that is capable of aggregating with other (like or different) components via hydrophobic interactions to form the multimolecular assembly may be utilized in accordance with the presently disclosed and/or claimed inventive concept(s). For example, but not by way of limitation, each of the plurality of components present in the multimolecular assembly may be a lipid, a fatty acid, an alkyl chain, a hydrocarbon-rich group, a fluorocarbon-rich group, a sterol, or any combination thereof. Particular non-limiting examples of components that may be utilized include a palmitoyl chain, an oleoyl chain, a stearoyl chain, an alkyl chain with 8 to 20 carbons and zero to multiple unsaturated bonds, a chain with 2 to 20 carbons with multiple fluorine atoms, and any combination thereof.

The heparosan polymer may be characterized as being substantially non-antigenic, substantially non-immunogenic, and substantially biologically inert within extracellular compartments of a mammalian patient, being stable in the mammalian bloodstream, and being degraded intracellularly in the mammalian patient. The heparosan polymer may be produced by any method known in the art or otherwise contemplated herein, as will be discussed in greater detail herein below. In addition, one of the advantages of the presently disclosed and/or claimed inventive concept(s) is that the heparosan can be synthesized in a step-wise, reproducible, and defined manner so as to provide all of the advantages of PEG without its potential side effects.

Any size and/or size distribution of heparosan can be utilized herein, so long as the heparosan can be conjugated to a hydrophobic molecule and function in accordance with the presently disclosed and/or claimed inventive concept(s). In addition, the heparosan polymer may be a linear chain or have a branched geometry. Alternatively, the heparosan polymer may have a dendritic geometry. The heparosan polymer will be unsulfated and unepimerized.

In certain non-limiting embodiments, the heparosan polymer may have a mass in a range of from about 600 Da to about 300 kDa. In addition, when multiple heparosan polymers are present on the surface of the multimolecular assembly, the plurality of heparosan polymers may be polydisperse or substantially monodisperse in size. In one non-limiting example, polymers that are “substantially monodisperse” in size are defined as: (a) having a molecular weight in a range of from about 3.5 kDa to about 300 kDa and a polydispersity value in a range of from about 1.0 to about 1.1; (b) having a molecular weight in a range of from about 0.5 MDa to about 300 kDa and a polydispersity value in a range of from about 1.0 to about 1.5; and/or (c) having a molecular weight in a range of from about 0.5 MDa to about 300 kDa and a polydispersity value in a range of from about 1.0 to about 1.2.

As described herein above, the heparosan (HEP) polymer may be derived by any method known in the art or otherwise contemplated herein, such as, but not limited to: (i) chemoenzymatic polymerization in vitro or (ii) microbial fermentation in vivo. In addition, the heparosan polymer may be attached to the hydrophobic moiety-containing component by any method known in the art or otherwise contemplated herein. For example, but not by way of limitation, the heparosan polymer may be provided with an activated group thereon that effects the covalent or non-covalent conjugation of the heparosan polymer to the hydrophobic moiety-containing component. Non-limiting examples of reactive groups that may be utilized in this manner include an aldehyde, alkyne, ketone, maleimide, thiol, azide, amino, carbonyl, sulfhydryl, hydrazide, phosphate, sulfate, nitrate, carbonate, ester, squarate, chelator, and any combination thereof. The heparosan polymer may be chemically transformed or activated into different reactive species suitable for attachment to the hydrophobic moiety-containing component, and depending on the chemical group(s) employed, these modifications can be done at various steps (e.g., modification of the acceptor pre-polymerization, or post-polymerization, or a combination of both, as well as during the polymer fragmentation steps, etc.).

The use of the multimolecular assembly in the drug delivery compositions of the presently disclosed and/or claimed inventive concept(s) affects the storage, stability, longevity, and/or release of the therapeutic/potential therapeutic agent disposed therein. For example, the drug delivery composition produced in accordance with the presently disclosed and/or claimed inventive concept(s) may: (a) exhibit increased retention in blood and/or lymphatic circulation of a mammalian patient when compared to therapeutic/potential therapeutic agent alone; (b) exhibit reduced occurrence of accumulation in healthy organs and/or tissues of a mammalian patient when compared to therapeutic agent/potential therapeutic agent alone; and/or (c) exhibit higher accumulation in tumors and/or diseased tissues of a mammalian patient when compared to therapeutic agent/potential therapeutic agent alone.

Table 2 describes some non-limiting examples of combinations of multimolecular assembly/therapeutic agent cargo types that may be utilized in accordance with the presently disclosed and/or claimed inventive concept(s).

TABLE 2
Drug Delivery Platform and Drug Cargo Overview

| Carrier | Lipid Assembly | Drug Type |
|---|---|---|
| Category | (non-limiting examples) | (non-limiting examples) |
| HEPosome | liposome, vesicles, etc. | hydrophilic small |
| | | molecules, and |
| | | macromolecules, peptides, |
| | | polypeptides, |
| | | polynucleotides and |
| | | nucleotide, small |
| | | molecules, dyes, etc. |
| HEPcelle | Micelles, amphiphilic | hydrophobic drugs, |
| | nanoparticles, etc. | non-polar molecules, |
| | | small molecules, etc. |

Another embodiment of the presently disclosed and/or claimed inventive concept(s) is directed to a pharmaceutical composition comprising one or more of any of the drug delivery compositions described or otherwise contemplated herein above in combination with a pharmaceutically acceptable carrier and/or aqueous medium, as described in further detail herein below.

The drug delivery compositions of the presently disclosed and/or claimed inventive concept(s) may be designed for administration to a mammalian patient by any method known in the art, or any method disclosed herein after or otherwise contemplated herein. In one non-limiting embodiment, the method may involve the preparation of an injectable, pharmaceutically active drug delivery composition that is capable of circulating in a mammalian bloodstream.

Certain embodiments of the presently disclosed and/or claimed inventive concept(s) are directed to a method for preparing a pharmaceutically active drug delivery composition, wherein the drug delivery composition may be any of the drug delivery compositions described or otherwise contemplated herein.

In one non-limiting embodiment of the method, a plurality of components as described herein above (i.e., at least a portion of which comprises at least one hydrophobic moiety, and at least one of which having a heparosan polymer attached thereto), is reacted with at least one therapeutic/potential therapeutic agent under conditions sufficient to effect aggregation of the hydrophobic moieties via hydrophobic interactions to form at least one multimolecular assembly from the plurality of components. In addition, the reaction conditions are also sufficient to entrap, carry, and/or bind the at least one therapeutic/potential therapeutic agent within or to the multimolecular assembly. The multimolecular assembly thus formed also has at least one heparosan polymer attached to a surface thereof.

In another non-limiting embodiment of the method, the multimolecular assembly is first formed, and then the heparosan polymer is added thereto. In this embodiment of the method, a plurality of components as described herein above is reacted with at least one therapeutic/therapeutic agent under conditions sufficient to effect aggregation of the hydrophobic moieties via hydrophobic interactions to form at least one multimolecular assembly from the plurality of components. In addition, the reaction conditions are also sufficient to entrap, carry, and/or bind the at least one therapeutic/potential therapeutic agent within or to the multimolecular assembly. The multimolecular assembly so formed is then reacted with at least one heparosan polymer attached to a hydrophobic moiety-containing component, and the at least one hydrophobic moiety attached to the heparosan polymer partitions into the multimolecular assembly so that the heparosan polymer is attached to a surface of the multimolecular assembly.

Other non-limiting embodiments of the presently disclosed and/or claimed inventive concept(s) are directed to methods of use of any of the drug delivery compositions that include multimolecular asssemblies “decorated” with heparosan as disclosed or otherwise contemplated herein above. In the methods, a therapeutically effective amount of any of the compositions described or otherwise contemplated herein is administered to a mammalian patient so as to induce a therapeutic effect in the mammalian patient.

The drug delivery composition (i.e., the multimolecular assembly “decorated” with heparosan) may be administered, for example but not by way of limitation, parenterally, intraperitoneally, intraspinally, intravenously, intramuscularly, intravaginally, subcutaneously, intranasally, rectally, and/or intracerebrally. Dispersions of the drug-heparosan conjugate or HEP-drug delivery composition may be prepared in glycerol, liquid poly[ethylene glycols], and mixtures thereof, as well as in oils. Under ordinary conditions of storage and use, such preparations of drug delivery composition “decorated” with heparosan may also contain a preservative to prevent the growth of microorganisms.

Pharmaceutical compositions suitable for injection use include sterile aqueous solutions (where water soluble) or dispersions and sterile powders for the extemporaneous preparation of sterile injectable solutions or dispersions. When used for injection, the composition should be sterile and should be fluid to the extent that easy syringability exists. The compositions should also be stable under the conditions of manufacture and storage and should be preserved against the contaminating action of microorganisms such as bacteria and fungi. The drug delivery composition “decorated” with heparosan may be used in conjunction with a solvent or dispersion medium containing, for example but not by way of limitation, water, ethanol, poly-ol (i.e., glycerol, propylene glycol, and liquid poly[ethylene glycol], and the like), suitable mixtures thereof, vegetable oils, and combinations thereof.

The proper fluidity of the drug delivery composition “decorated” with heparosan may be maintained, for example but not by way of limitation, by the use of a coating such as lecithin, by the maintenance of the required particle size in the case of dispersion, and/or by the use of surfactants. Prevention of the action of microorganisms may be achieved by various antibacterial and antifungal agents, such as but not limited to, parabens, chlorobutanol, phenol, ascorbic acid, thimerosal, and the like. In many cases, it will be desirable to include isotonic agents, for example, sugars, sodium chloride, or polyalcohols such as mannitol and sorbitol, in the composition. Prolonged absorption of the injectable compositions may be brought about by including in the composition an agent that delays absorption, such as but not limited to, aluminum monostearate and/or gelatin.

Sterile injectable solutions may be prepared by incorporating the drug delivery composition “decorated” with heparosan in the required amount in an appropriate solvent with one or a combination of ingredients enumerated above, as required, followed by filtered sterilization. Generally, dispersions are prepared by incorporating the drug-heparosan conjugate or HEP-drug delivery composition into a sterile carrier that contains a basic dispersion medium and the required other ingredients from those enumerated above. In the case of sterile powders for the preparation of sterile injectable solutions, the methods of preparation may include vacuum drying, spray drying, spray freezing, and/or freeze-drying that yields a powder of the active ingredient (i.e., the drug-heparosan conjugate or HEP-drug delivery composition) plus any additional desired ingredient from a previously sterile-filtered solution thereof.

The drug delivery composition “decorated” with heparosan may be orally administered, such as but not limited to, with an inert diluent or an assimilable edible carrier. The drug delivery composition “decorated” with heparosan and other ingredients may also be enclosed in a hard or soft shell gelatin capsule, compressed into tablets, and/or incorporated directly into the subject's diet. For oral therapeutic administration, the drug-heparosan conjugate or HEP-drug delivery composition may be incorporated with excipients and used in the form of ingestible tablets, buccal tablets, troches, capsules, elixirs, suspensions, syrups, wafers, and the like. The percentage of the drug delivery composition “decorated” with heparosan in the compositions and preparations may, of course, be varied as will be known to the one of ordinary skill in the art. The amount of the drug delivery composition “decorated” with heparosan in such therapeutically useful compositions is such that a suitable dosage will be obtained.

In certain embodiments it may be desired to formulate parenteral compositions in dosage unit form for ease of administration and uniformity of dosage. The term “dosage unit form” as used herein refers to physically discrete units suited as unitary dosages for the subjects to be treated, with each unit containing a predetermined quantity of drug-heparosan conjugate or HEP-drug delivery composition calculated to produce the desired therapeutic effect. The specification for the dosage unit forms of the presently disclosed and/or claimed inventive concept(s) are dictated by and directly dependent on (a) the unique characteristics of the drug-heparosan conjugate or HEP-drug delivery composition and the particular therapeutic effect to be achieved, and (b) the limitations inherent in the art of compounding such a therapeutic compound for the treatment of a selected condition in a subject.

Incorporation of various HEP-HMs onto multimolecular assemblies (such as, but not limited to, liposomes, lipid vesicles, and/or aggregation of hydrophobic heparosan into micelles or lipid aggregates) are demonstrated herein using various hydrophobic molecules and multimolecular assembly structures. Various sizes of quasi-monodisperse heparosans were coupled to the hydrophobic molecules using various conjugation chemistries. The resulting HEP-multimolecular assembly drug delivery compositions (i.e., hydrophobic heparosan derivative incorporated into multimolecular assemblies such as, but not limited to, liposomes (“HEPosomes”) and/or in micelles (“HEPcelles”)) were detected via electrophoresis and/or chromatographic methods. The HEPosomes and HEPcelles retained drugs or other molecules, as observed in various in vivo and in vitro assays.

As described in detail herein after, multiple types of hydrophobic molecules (HM), including fatty acids, phospholipids, and cholesterol, have been conjugated to the heparosan (HEP) polysaccharide vehicle. The HM serves as an anchor that can bind or immobilize the hydrophilic heparosan chain (i.e., a carbohydrate with numerous polar hydroxyls and carboxylates) to the multimolecular assembly (i.e., liposome, micelle, etc.) via interaction with its hydrophobic components (e.g., acyl chains in a bilayer, non-polar core moieties, etc.). Various coupling chemistries were used to link groups on the hydrophobic molecules to groups on the heparosan vehicle (functionalities including, but not limited to, amines, aldehydes, sulfhydryls, maleimides, squarates, iodoacetyl, etc.). Various sizes (i.e., different polymer chain lengths; measured in kDa=thousands of Dalton; including approximately 700 Da to approximately 300 kDa range) of quasi-monodisperse (narrow size distribution) heparosan were coupled to the hydrophobic molecules forming various HEP-HM conjugates.

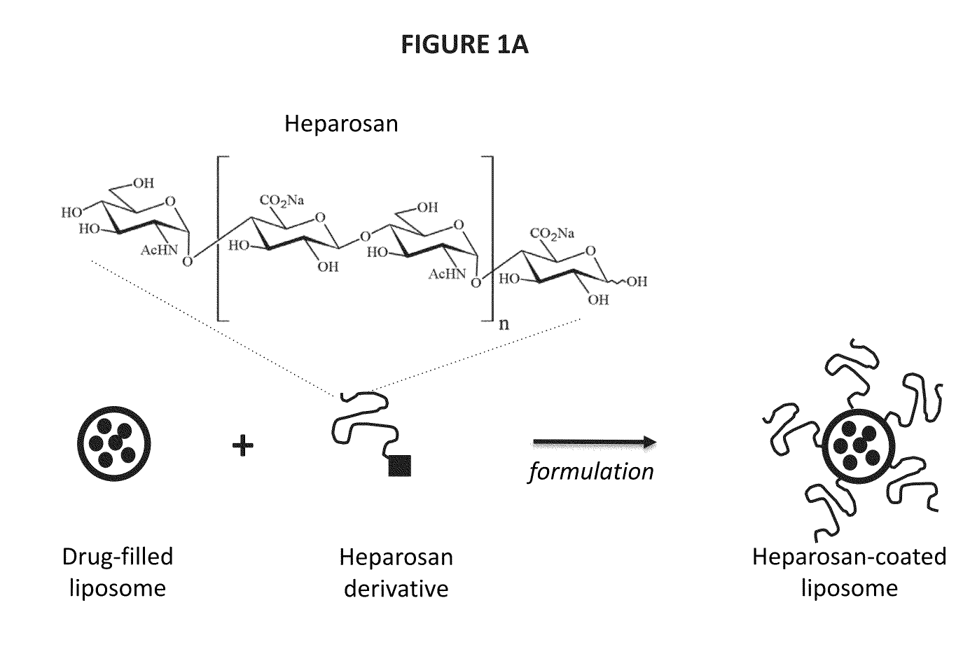

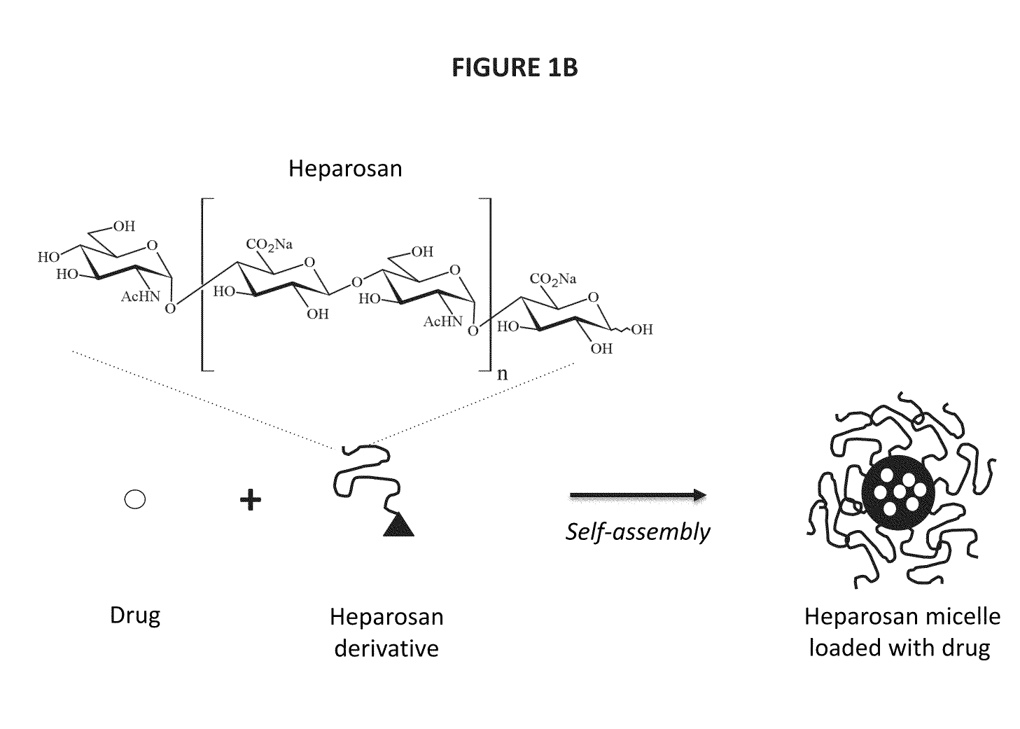

The HEP-HM conjugates were incorporated into multimolecular assemblies (i.e., liposomes, micelles, or similar nanoparticle assemblies), including multimolecular assemblies that contain entrapped molecules (e.g., drugs, drug candidates, or therapeutic small molecular weight compounds or macromolecules); in general, this assembly includes various combinations of at least two of the group of: (a) HEP-HM, (b) amphiphilic molecules, and (c) therapeutics, and is here termed a “HEPosome” or a “HEPcelle” (liposomes or micelles, respectively; when in typical aqueous systems, the former have an aqueous interior, while the latter have a hydrophobic core; see FIGS. 1A and 1B). Several methods of synthesis are possible, including but not limited to, where the HEP-HM is: (i) added to the multimolecular assemblies after they are formed (post-modification), or (ii) during the initial or the intermediate phase(s) of production process at the start. Importantly, the addition of the HEP-HM molecules to the multimolecular assembly as in method (i) was achieved with retention of integrity, and no detergent-like disruption or significant release of entrapped agent was noted.

The HEPosome (FIG. 1A) was also protected by virtue of its heparosan coating in a model system from destruction by pore-forming toxins that are similar biophysically to the attacks mediated by the mammalian patient, such as the complement system. HEPcelles (FIG. 1B) are self-assembling aggregates containing a HEP-HM that may or may not have an additional amphiphilic component. In another embodiment, the therapeutic/potential therapeutic agent itself could serve as the hydrophobic component (i.e., the HM) of the HEPcelle amphiphilic structure.

The sugar chains decorating the outside of the multimolecular assembly provide a surface that will allow longer half-lives in the circulation compared to the unmodified multimolecular assembly (i.e., without heparosan on a surface thereof). Such platforms are suitable for carrying a plethora of therapeutics/potential therapeutics, including for example but not by way of limitation, many macromolecular drugs (e.g., proteins, polynucleotides, etc.), chemotherapy drugs, steroids, antibiotics, nutrients, small molecules, and the like.

As discussed herein above, the pharma industry has employed a variety of drug delivery strategies, and the presently disclosed and/or claimed inventive concept(s) improves upon these current strategies by focusing on multimolecular assemblies that utilize heparosan. The HEP molecule, the biosynthetic precursor to the well-known drug heparin and to the widespread glyco-moiety heparin sulfate (HS) in the body, appears tolerated due to its ‘self’ nature as well as its intrinsically favorable behavior in the bloodstream and tissues. The polysaccharide is stable in the extracellular spaces of mammals, but degraded by lysosomal enzymes following entry into the cell. Heparosan is predicted to serve as a protective agent to the delivery of therapeutics without liabilities of polydispersity, immunogenicity, and/or unwanted accumulation in the body that are observed for other types of polymers, such as but not limited to, poly(ethylene glycol), hydroxyethyl starch, or poly(sialic acid).

From a chemophysical standpoint, the heparosan chain is very hydrophilic due to its two hydroxyl groups on every monosaccharide unit and a negative carboxylate group on every other monosaccharide unit (FIG. 1A). Heparosan should be biocompatible in the human body because it is the endogenous natural precursor in the heparin/heparan sulfate (HS) biosynthetic pathway (Sugahara et al., 2002). Stretches of heparosan exist in the heparin/HS chains found on virtually every human cell; thus, it is not perceived as a foreign molecule. Certain pathogenic bacteria even exploit the “self” nature of heparosan by using a heparosan coating or capsule to evade the immune system during infection. In the pre-genomic era, the difficulty in raising antibodies to heparosan for use as ‘typing sera’ was exemplified by the need to use either a capsule-specific bacteriophage (a virus) or bacterial heparin-degrading enzymes to make these microbial identifications.

In contrast to HS and heparin, the heparosan molecule is neither decorated with sulfate groups nor epimerized at glucuronic acid residues; thus, heparosan is biologically inactive to a significant extent with respect to coagulation (i.e., does not activate clotting factors to a significant extent), modulation of proliferation (i.e., does not bind to growth factors at a significant extent), inflammation (i.e., does not interact with cytokines at a significant extent), and a plethora of other activities (Capila et al., 2002). Furthermore, enzymes that degrade heparin or HS (i.e., heparanase) or receptors that clear HS from the bloodstream (i.e., Hyaluronan Receptor for Endocytosis (HARE) or stabilin) do not recognize heparosan to a significant extent, because the sulfate groups essential for activity are absent from this polymer (Pikas et al., 1998; and Harris et al., 2008). In other words, heparosan reads as ‘a hole in the sugar code’ that is ignored or is not significantly affected by the HS recognition systems. Therefore, heparosan is stable in the extracellular spaces where many therapeutic drugs act or are delivered. Over time, bulk cellular fluid uptake or pinocytosis will internalize heparosan, but once the polymer arrives at the lysosome, the normal degradation pathway for HS/heparin removes sugars from the non-reducing end in a sequential fashion. In contrast, PEG is not degraded in such a fashion; larger PEG is particularly long lasting in the body; thus, most PEGylation reagents are in the 5- to 40-kDa range (much smaller than the largest HEP reagents possible) to avoid accumulation in the tissues. Similarly, HES reagents are usually below 70 kDa.

The theoretical predictions for the behavior of heparosan in the body have been borne out by various experimental tests in rodents and primates (U.S. Published Patent Application No. 2010/0036001, published by DeAngelis on Feb. 11, 2010; the entire contents of which are hereby expressly incorporated herein by reference). The molecule has about a 0.5-day to 8-day half-life in the bloodstream, depending on its molecular weight and the route of injection. The polymer chain length is stable in plasma. No significant accumulation in tissues was observed. Over time the metabolites of the probe linker with short sugar chain are excreted in urine and feces. The polymer does not cross the blood-brain barrier to a significant extent, which may be important for ensuring the safety of some therapeutics. No detrimental toxicological effects were observed with acute doses of 100 milligram per kilogram (mg/kg) in a rodent model.

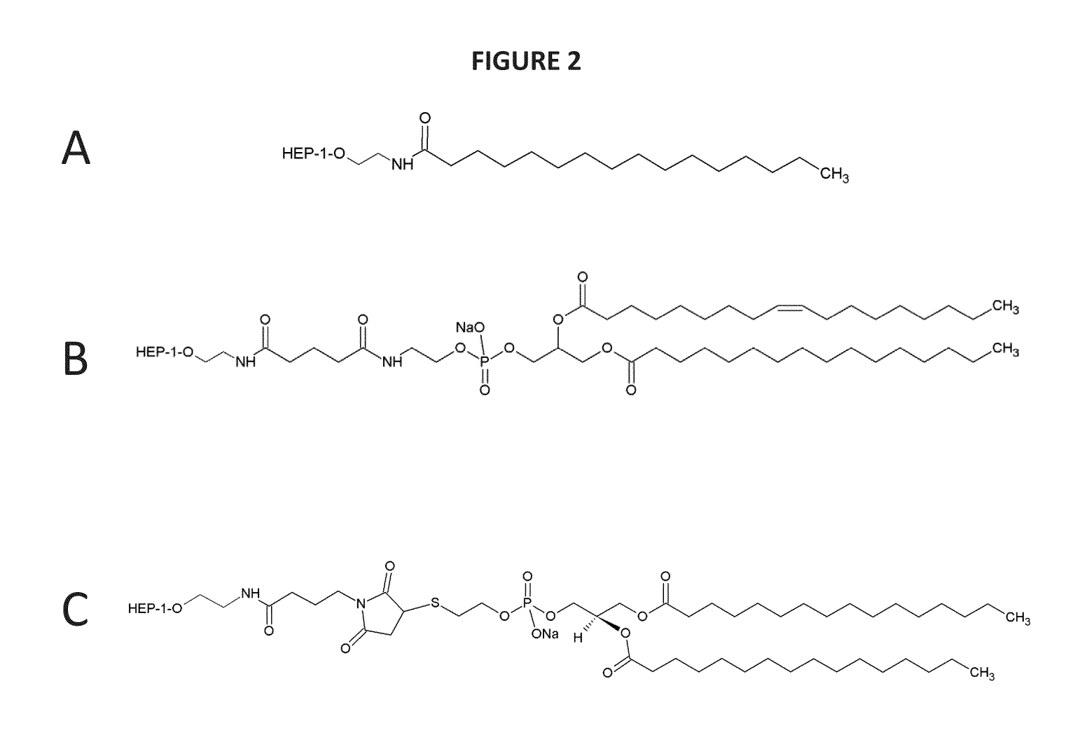

Most of the typical chemistries used to modify molecules with PEG are amenable to use with heparosan. For example, reactive heparosan polymers with a single amine-reactive functionality (e.g., aldehyde, squarate), sulfhydryl-reactive functionality (e.g., maleimide, iodoacetyl), or carbonyl-reactive functionality (e.g., amine) at the reducing terminus have been conjugated to various hydrophobic molecules that have affinity for liposomes, or may be used to make liposomes or micelles either as a pure component or as mixtures with other amphipathic molecules. Non-limiting examples of structures for such HEP-HMs are shown in FIG. 2A-F. This listing of chemical coupling methods to produce hydrophobic conjugates is not exhaustive; the spirit of the presently disclosed and/or claimed inventive concept(s) includes any conjugate made by covalently bonding heparosan polymer(s) to a molecule that can be used in creating liposomal or micellar drug delivery platforms. One basic inventive concept is the addition of HEP to the hydrophobic anchoring molecule to enhance therapeutic performance of the drug delivery composition in the body and improve patient well-being. Another basic inventive concept is the addition of HEP to the relatively hydrophobic drug or drug candidate molecule (e.g., thus forming a micelle or nanoparticle) to enhance therapeutic performance in the body and improve patient well-being.

In addition to the ‘biological’ issues surrounding the drug's behavior in the body, the heparosan polymer should also be amenable to facilitate manufacture and quality control. Therefore, the polymer size distribution (polydispersity) should be narrow such that the final drug delivery platform will have uniform effectiveness. In addition, the efficiency of coupling to the anchor should be as high as possible; in one particular case, at least about 25% to about 100% of the anchor should be modified in a coupling reaction, and in another particular case, about 80% to about 100% should be modified in a coupling reaction. PEG, with a longer history of use and simpler chemical synthesis, has addressed the issues of polydispersity and activation to a large degree, but the HES and PSA carbohydrate systems are still somewhat lacking in these regards.

From a manufacturing perspective, mammalian HS or heparin is not the ideal starting material for heparosan, because chemical transformation by desulfation is problematic and will always result in damaged backbone chains and/or residual sulfate groups. In addition, animal-derived materials are not perceived favorably by drug regulatory agencies due to the potential risk of contamination by adventitious agents (e.g., prions, virus, etc.). The human enzymes that form heparosan by polymerizing the uridine diphospho-sugar (UDP-sugar) precursors in the HS biosynthetic pathway are also non-ideal; they are weakly expressed in recombinant systems and are poor catalysts in vitro. Fortunately, certain pathogenic bacteria possess very useful enzymes that produce heparosan (DeAngelis, 2002). One such enzyme, PmHS1, the heparosan synthase from Pasteurella multocida, is very active and stable in recombinant forms (including, but not limited to, fusion proteins, truncations, mutants, analogs, and combinations thereof) (Sismey-Ragatz et al., 2007). In addition, PmHS2 or chimeric recombinant enzyme versions that combine the activities of PmHS1 and PmHS2 can be used with varying levels of efficiency.

In one embodiment, heparosan manufacture utilizes a novel synchronized, stoichiometrically-controlled reaction employing a sugar-polymerizing enzyme (e.g., PmHS1, PmHS2, or combinations or similar analogs thereof with roughly equivalent biological activity) in an aqueous buffer system that results in a quasi-monodisperse (very narrow size distribution) product. The heparosan synthase can be utilized in vitro to synthesize quasi-monodisperse (i.e., very narrow size distributions approaching the ideal polydispersity value of ‘1’) polymer preparations (Sismey-Ragatz et al., 2007) with homogenous reactive end-groups for coupling to biologic targets. The narrow size distribution is achieved by synchronizing the polymerization reaction using a primer, a short heparosan fragment, which allows the normal slow chain initiation step of biosynthesis to be bypassed. Therefore, all polymers are rapidly extended by PmHS1 in a virtually parallel fashion; thus, all final chains have a very similar length. No post-polymerization purification for size control is required. The primer also contributes the unique reactive group(s) that helps assure that every polymer chain can be activated for anchor coupling. In addition, the primer position in the heparosan chain at the reducing terminus does not interfere with lysosomal degradation, thus allowing the heparosan chain to be digested to a tiny stub containing the linker site used for anchor attachment; this stub is usually excreted in the urine or feces.

The chain size or molecular weight of any particular heparosan preparation is controlled by manipulating the stoichiometric ratio of the primer to the UDP-sugar precursor. Basically, for a given amount of UDP-sugars, a low concentration of primer yields longer chains while, on the other hand, a high primer concentration yields shorter chains (of course, the former case has fewer moles of product formed than the latter). Heparosan molecules in the range of from about 10 kDa to about 4,500 kDa (or from about 50 to about 22,500 monosaccharide units) have been synthesized by the synchronized chemoenzymatic method, thus potentially accessing a wider useful size range than possible for PSA, HES, or PEG. By using other methods such as step-wise synthesis (elongation with a pair of PmHS1 mutants) or chemical fragmentation, heparosan polymers from about 600 Da to about 100 kDa and larger can be made.

The heparosan chemoenzymatic process has been run at the 150-gram level while retaining the same polydispersity values (about 1.003 to about 1.2, depending on chain size) as observed in the milligram level syntheses (Sismey-Ragatz et al., 2007); thus, scaling to the kilogram production level by this method is possible. The polymer uniformity facilitates production and quality control aspects essential for drug approval. Typical polydispersity values are in a range of from about 1.0 to about 1.1.

In addition to in vitro chemoenzymatically produced heparosan, the same [−4-N-acetylglucosamine-α1,4-glucuronic acid-β1-]ₙ polymeric structure may also be produced in vivo by the culture or fermentation of certain microbes; however, the polymer produced in this manner may not be as monodisperse or as easily activated for coupling in comparison to the in vitro produced polymer. Some examples of the fermentation systems include natural heparosan producers such as, but not limited to, Pasteurella multocida or allies (e.g., Avibacterium species), Escherichia coli K5, or the recombinant versions (e.g., Gram− or Gram+ bacteria, Achaea, or eukaryotic hosts) expressing the heparosan biosynthetic machinery (e.g., synthases, polymerases, or glycosyltransferases, UDP-glucose dehydrogenases, etc.) of the natural heparosan-producing species. The microbial heparosan production route may be used to make polymers for preparation of HEP-HM conjugates, and is covered by the spirit and scope of the presently disclosed and/or claimed inventive concept(s); the source of heparosan polymer is not important for construction of delivery platforms employed for the enhancement of therapeutic efficacy.

Heparosan meets the criteria for a desirable drug delivery component on multiple fronts. From an intrinsic point of view, heparosan has an approximately 500 million year safety profile; all animals from hydras to humans synthesize and display HS (with its endogenous stretches of heparosan) on their cell surface. In the simplest embodiment of modification, the geometry of attachment to the anchor is also identical to the natural proteoglycans (i.e., heparin sulfate post-translationally modified glycoproteins), where the reducing end of the heparosan chain(s) is attached to the polypeptide chain.

Furthermore, metazoan cells intracellularly metabolize heparosan along with heparan sulfate and heparin, and the chain is degraded from the non-reducing end. Therefore, in the simplest embodiment of the heparosan-based technology, after lysosomal processing only a short stub composed of the synthetic linker (the attachment site to HM anchor) and 1-3 monosaccharides is excreted from the body, in a similar fashion to the pathways for many small molecular weight drugs, hormones, and hydrophobic molecules. This scenario is a major improvement over PEG, where the lack of a natural degradation and excretion pathway contributes to accumulation of the unnatural polymer in tissues.

As stated herein above, the addition of heparosan (HEP) to an anchor molecule is superior to PEGylation because: a) heparosan has a higher water solubility than PEG; b) as a naturally occurring polysaccharide, heparosan's degradation products are biocompatible; and c) heparosan is not immunogenic.

In humans, polymers of pure heparosan only exist transiently, serving as a precursor to the more highly modified final products of heparan sulfate and heparin. The bacterial-derived enzymes used to produce heparosan for use in one embodiment of the presently disclosed and/or claimed inventive concept(s) synthesize heparosan as their final product. A single polypeptide, the heparosan synthase PmHS1 of Pasteurella multocida Type D, polymerizes the heparosan sugar chain by transferring both GlcUA and GlcNAc. PmHS1 is a robust enzyme that efficiently makes polymers up to approximately about 4.5 MDa (i.e., about 4,500 kDa or about 22,000 monosaccharide units) in vitro. In Escherichia coli K5, at least two enzymes, KfiA, the alpha-GlcNAc transferase, and KfiC, the beta-GlcUA-transferase (and perhaps KfiB, a protein of unknown function), work in concert to form the disaccharide repeat of heparosan. The E. coli enzyme complex is not as efficient as the PmHS1 enzyme, as it is more difficult to produce the long polymer chains with the E. coli enzyme complex. However, for the purpose of the presently disclosed and/or claimed inventive concept(s), it is intended and will be understood that any heparosan production method known or otherwise contemplated in the art falls within the scope of the presently disclosed and/or claimed inventive concept(s). It is not the method of producing heparosan that is determinative—rather, it is the conjugation of heparosan from any source or method of production (e.g., fermented heparosan produced by native or recombinant microbes, as well as chemoenzymatic syntheses or organic chemical syntheses) to a target molecule (i.e., the anchor) for better performance of a drug delivery platform within the patient that is presently disclosed and/or claimed.

A key advantage to using heparosan is that it has increased biostability in the extracellular matrix when compared to other glycosaminoglycans (GAGs) such as hyaluronic acid and chondroitin. As with most compounds synthesized in the body, new molecules are typically made, and after serving their purpose, are broken down into smaller constituents for recycling.

Heparin and heparan sulfate, for example, are degraded by a single enzyme known as heparanase. Experimental challenge of heparosan and N-sulfo-heparosan with heparanase, however, shows that since these polymers lack the O-sulfation of heparin and heparan sulfate, heparosan and N-sulfo-heparosan are not sensitive to enzymatic action in vitro by heparanase. These findings indicate that heparosan is not fragmented enzymatically in the body, thereby indicating that heparosan is a stable biomaterial for use in lipid-based or amphipathic carrier platforms.

However, if heparosan or any of its fragments (generated by reactive oxygen species, etc.) is internalized into the lysosome, then the molecules will be degraded by resident beta-glucosidase and alpha-hexosaminidase enzymes (which remove one sugar at a time from the non-reducing termini of the GAG chain), similar to the degradation of heparin or hyaluronic acid. Therefore, the heparosan polymer is biodegradable and will not permanently reside in the body and thereby cause a lysosomal storage problem. A key advantage for therapeutic modification with a heparosan polymer is that normal monosaccharides, GlcNAc and GlcUA, are the products of the eventual degradation. In contrast, PEG degrades into reactive artificial aldehydes and ketones which are toxic above certain levels. PEG also accumulates in the body, especially when present as one or more high molecular weight polymers.

The normal roles of heparin/heparan sulfate in vertebrates include binding coagulation factors (to inhibit blood clotting) and growth factors (to signal cells to proliferate or differentiate). The key structures of heparin/heparan sulfate that are recognized by these factors include a variety of O-sulfation patterns and the presence of iduronic acid [IdoUA]; in general, polymers without these modifications do not stimulate clotting or cell growth. Heparosan-based materials which do not have such O-sulfation patterns, therefore, do not provoke unwanted clotting or cellular growth/modulation. As such, heparosan-anchor conjugates do not initiate clotting and/or cell growth processes and remain solely bio-reactive as per the drug or cargo constituent carried in the liposomal or micellar system; thus, the heparosan is termed or deemed to be biologically inert.

Foreign or unnatural molecules stimulate the immune system. Heparosan polymer exists transiently during heparan sulfate and heparin biosynthesis as well as being found in very short polymer structures within mature heparan sulfate or heparin chains. In the latter case, the N- and O-sulfation reactions are not complete in mammals, so traces of the original heparosan remain; for example, approximately 1-5 unsulfated disaccharide repeats can be interspersed within the sulfated regions. Therefore, the body treats heparosan as ‘self,’ and does not mount an immune response. P. multocida Type D and E. coli K5 utilize heparosan coatings to ward off host defenses by acting as molecular camouflage. Indeed, scientists had to resort to using capsule-specific phages or selective GAG-degrading enzymes to type these heparosan-coated microbes, since a conventional antibody or serum could not be generated; thus, the heparosan is termed or deemed non-immunogenic or non-antigenic.

### EXAMPLES

Examples are provided hereinbelow. However, the presently disclosed and claimed inventive concept(s) is to be understood to not be limited in its application to the specific experimentation, results and laboratory procedures. Rather, the Examples are simply provided as one of various embodiments and are meant to be exemplary, not exhaustive.

### Example 1

### Heparosan-Hydrophobic Molecule (HEP-HM) Reagents

Various couplings of HEP and HM are described below. In some cases, quasi-monodisperse heparosan containing one primary amine linker at the reducing end of each polymer chain (synthesized by chemoenzymatic polymerization using PmHS1, oligosaccharide acceptor with a free amine, and UDP-sugars, as covered by U.S. Pat. No. 8,088,604, incorporated supra) were conjugated directly to hydrophobic molecules containing an amine-reactive group. In some cases, an intermediate activation step was used in the conjugation chemistry between heparosan polymers and the hydrophobic molecules; for example, but not by way of limitation, an intermediate heterobifunctional reagent was reacted with the amine to allow introduction of a new coupling functionality such as a maleimide, squarate, etc.

While particular examples demonstrate modification of the reducing terminal of the HEP chain with the HM, it will be understood that this attachment position is not to be considered limiting. For example, the HM moiety could be attached along the backbone (e.g, on the glucuronic acid groups, hydroxyls, or a de-amidated GlcNAc) or to the non-reducing termini and still be used in the various drug delivery platforms described or otherwise contemplated therein. Any attachment point(s) can be utilized so long as: (a) the presence of covalent modifications does not significantly hinder or block the normal lysosomal degradation pathway (i.e., sequential exoglycosidase action from the non-reducing termini), (b) unnatural epitopes are not produced that may spawn immunogenicity, and (iii) a heterogeneous product (different levels and/or locations of modification) is not produced, unless specifically desired.

### I. Chemical Activation of Heparosan

### HEP-NH₂ Polymer with N-hydroxysuccinimide or sulfo N-hydroxysuccinimde esters

The amine group is a useful functionality for further chemical modification, but the natural heparosan [−4-N-acetylglucosamine-α1,4-glucuronic acid-β1-] structure does not normally contain a free amine; therefore, such a functionality or reactive group must be placed in a desirable position by the hand of man. Heparosan with a free amino group at its reducing terminus can be prepared by multiple routes including, but not limited to: (i) chemoenzymatic extension of heparosan oligosaccharides containing an amine (or an amine intermediary or precursor, e.g., azido, etc.) using UDP-sugars and a heparosan synthase; (ii) nitrous acid fragmentation of heparosan polymer and reductive amination with a diamine or ammonia; or (iii) reductive amination of heparosan with a diamine or ammonia. Modification at the reducing termini of the HEP chain does not interfere with the normal lysosomal processing of heparosan that is mediated by exoglycosidases that act at the non-reducing terminus; thus, this modification of the reducing termini of heparosan is particularly well suited for making HEP-HM conjugates that are biodegradable.

In certain non-limiting embodiments of the method, the primary amine group at the reducing termini of the heparosan-NH₂ polymer is converted into a thioacetyl, iodoacetyl, maleimide, or aldehyde group or any other desired functionality that is compatible with the reaction conditions; this conversion is achieved by treating the polymer with the appropriate heterobifunctional reagent (i.e., a reagent with two distinct chemically reactive moieties). Typically, such heterobifunctional reagents possess (but are not limited to possessing): (i) an N-hydroxysuccinimide or sulfo-N-hydroxysuccinimide ester (e.g., NHS-esters); and (ii) the desired thioacetyl, iodoacetyl, maleimide, or aldehyde group, etc. In such an activation reaction, the heparosan amine reacts with the NHS-ester of the heterobifunctional reagent, forming a stable amide bond, and the other desired group is now added to the reducing terminus of heparosan chain; this desired group is later coupled to the therapeutic/potential therapeutic agent.

In one particular, non-limiting example, heparosan-NH₂ at a concentration of approximately 5-40 mg/mL in a solution of 100 mM sodium phosphate, pH 6.5-8.5 (or similar neutral to basic pH buffer that lacks free amino groups), and further containing 0-35% dimethylsulfoxide (if needed for solubility of the activation reagent) was reacted with approximately 2-50 molar equivalents of suitable N-hydroxysuccinimide or sulfo-N-hydroxysuccinimide esters containing the desired functionalities at 4-37° C. for 1-12 hours (also, modification of this general protocol to some degree is well within the skill of those of ordinary skill in the art). The obtained polymers can be recovered by various methods well known in the art including, but not limited to, precipitation with a water-mixable organic solvent (such as, but not limited to, alcohols or acetone), anion exchange chromatography, size exclusion chromatography, tangential flow filtration, or ultrafiltration. In addition, any combination of these recovery techniques can be also used to obtain purified activated polymer.

### II. Chemical Activation of Heparosan

### HEP-NH₂ Polymer with 3,4-diethoxy-3-cyclobutene-1,2-dione

Alternatively, instead of employing a NHS-ester to activate heparosan-NH₂, one can employ squarene-based (“squarate”) reagents (e.g., 3,4-diethoxy-3-cyclobutene-1,2-dione) as an intermediate or the end product. In one non-limiting example, the reducing termini primary amine of heparosan-NH₂ (approximately 5-40 mg/mL) in 50-100 mM sodium phosphate, pH 7 to 7.5 (or similar neutral to basic pH buffer that lacks free amino groups), and 15-30% ethanol was converted into the mono-squaramide by treatment with approximately 2-100 molar equivalents of 3,4-diethoxy-3-cyclobutene-1,2-dione for 1-12 hours.

The obtained polymers can be recovered by various methods well known in the art including, but not limited to, precipitation with a water-mixable organic solvent (such as, but not limited to, alcohols or acetone), anion exchange chromatography, size exclusion chromatography, tangential flow filtration, or ultrafiltration. In addition, any combination of these recovery techniques can be also used to obtain purified activated polymer.

### III. Chemical Activation of Heparosan

### Branched HEP-Aldehyde Polymers

In another non-limiting example, an aldehyde-containing (e.g., an amino reactive group) branched (i.e., containing multiple chains with a common attachment site) heparosan moiety was synthesized. First, tris(2-aminoethyl)amine hydrochloride was reacted with 1 molar equivalent of succinimidyl-p-formylbenzoate in 50 mM HEPES in 75% dimethylsulfoxide at pH 7.0 for 3.5 hours at ambient temperature. The reaction mixture was diluted with 20 volume equivalents of water and filtered, and the filtrate was evaporated to dryness. Cation exchange chromatography over HiTrap® SP chromatography columns (GE Healthcare Bio-Sciences AB LLC, Uppsala, Sweden) at pH 4.0 with a sodium chloride gradient yielded N-{2-[bis(2-aminoethyl)amino]ethyl}-4-formylbenzamide, which was subsequently desalted by reversed phase solid phase extraction over STRATA™-X SPE tubes (Phenomenex, Inc., Torrance, Calif.). Second, N-{2-[bis(2-aminoethyl)amino]ethyl}-4-formylbenzamide was reacted with 10 molar equivalents of succinimidyl acetylthioacetate in 35 mM HEPES buffer, pH 7.0, in 66.6% dimethylsulfoxide at ambient temperature for 3 hours, after which time an additional 6 molar equivalents of succinimidyl acetylthioacetate in dimethylsulfoxide were added, and the reaction left to continue for another 2.5 hours. The mixture was freeze-dried overnight, and the obtained residue was purified by reversed phase chromatography to yield the target crosslinker with two thioacetate and one benzaldehyde functionalities. Third, the two thioacetate functionalities were reacted with 2.5 molar equivalents of a 41.5-kDa heparosan-maleimide polymer in 100 mM sodium phosphate, pH 7.0, 50 mM hydroxylamine hydrochloride, and 2.5 mM EDTA overnight at ambient temperature. However, other buffer systems in the pH range from 6.5-8.5 will also work. The obtained dimer was separated from unreacted monomer by size-exclusion chromatography over a SEPHACRYL® S-300 column (GE Healthcare Bio-Sciences, Pittsburgh, Pa.) with 50 mM sodium phosphate, pH 7.0, 150 mM NaCl as eluent. The combined clean dimer-containing fractions were precipitated by addition of isopropanol, dried in vacuo, and re-dissolved in water to yield the target heparosan dimer with a reactive aldehyde functionality at the reducing end.

It is to be understood that the synthesis of the particular reagent described herein above is simply for purposes of illustration only. Any desired activated branched heparosan reagent having other architectures (e.g., more branches, etc.) or reducing terminal reactive groups (e.g., maleimide, amine, iodoacetate, squarate) that can function in accordance with the presently disclosed and/or claimed inventive concept(s) may be synthesized by similar strategies and routes of synthesis.

### Example 2

### Heparosan Conjugation To Hydrophobic Molecules

In this Example, syntheses of various HEP-HM (hydrophobic moiety) derivatives are described. Table 3 provides an overview of some examples of the hydrophobic molecules and chemistries that can be used to modify HEP into HEP-HM. However, these particular hydrophobic molecules and chemistries are not to be considered limiting; other hydrophobic molecules and chemistries that can be used in accordance with the presently disclosed and/or claimed inventive concept(s) are easily identifiable by those of ordinary skill in the art.

TABLE 3
Hydrophobic Molecule Overview and Non-Limiting Examples

| | | Functionality | |
|---|---|---|---|
| | Functionality | of the target | |
| | on the | reactive group | |
| “Name of Hydrophobic Moiety (HM)” | hydrophobic | (HEP reactive | |
| Hydrophobic molecule | molecule | group) | HEP size (kDa) |
| “Palmitoyl” | | | |
| Palmitic acid N-hydroxysuccinimide ester | NHS-ester | Amine | 0.8, 12, 26 & 75 kDa |
| Palmitic anhydride | Anhydride | Amine | 29 kDa |
| “Dipalmitoyl”—1,2-Dipalmitoyl-sn- | Thiol | Maleimide, | 12 and 42 kDa |
| Glycero-3-Phosphothioethanol | | Iodoacetyl | |
| “Palmitoyl/Oleoyl”—N-(Succinimidyloxy- | NHS-ester | Amine | 26 kDa |
| glutaryl)-L-α-phosphatidylethanolamine, 1- | | | |
| Palmitoyl-2-oleoyl | | | |
| “Cholesterol”—Thiocholesterol | Thiol | Maleimide, | 13 kDa |
| | | Iodoacetyl | |
| “Distearoyl”—1,2-Distearoyl-sn-glycero- | Amine | Squarate | 26 and 75 kDa |
| 3-phosphoethanolamine | | | |
| “Fluoroalkyl”—Heptadecafluoroundecyl | Maleimide | Sulfhydryl | 29 kDa |

### I. Synthesis of HEP-Palmitoyl Derivative (FIG. 2A)

In one embodiment, essentially quasi-monodisperse heparosan containing one primary amine linker at the reducing end of each polymer chain (synthesized by chemoenzymatic polymerization using PmHS1, oligosaccharide acceptor and UDP-sugars as covered by U.S. Pat. No. 8,088,604, incorporated supra) was reacted with 50-300 molar equivalents of N-succinimidyl palmitate in a 96% aqueous dimethylsulfoxide solution containing 40 mM sodium phosphate, pH 7.0, at ambient temperature for 16 hours. However, N-succinimidyl esters of other fatty acids, as well as different solvent systems, solvent ratios, buffers, pH ranges, temperatures, and reaction times can equally be employed to synthesize conjugates between heparosan and fatty acids; the selection of the different variables is well within the skill of one of ordinary skill in the art.

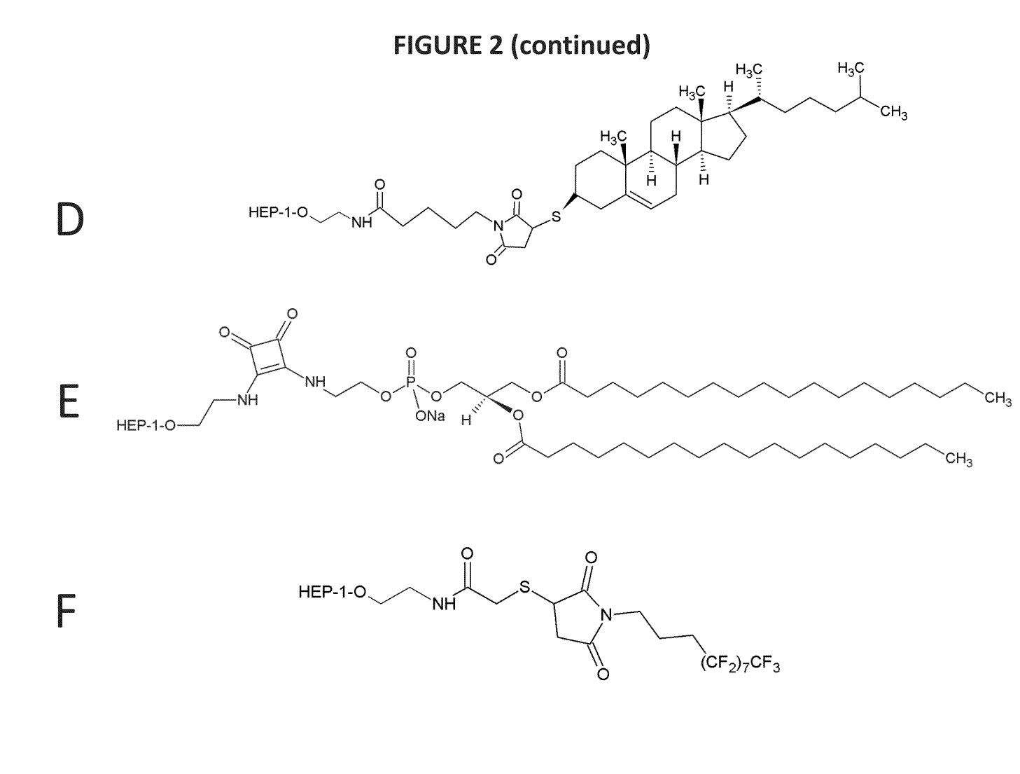

The obtained reaction mixture was purified by precipitation with isopropyl alcohol (although other water-miscible organic solvents such as acetone or ethanol can also be employed) and/or anion exchange chromatography. The purified conjugate (C) was quantified by carbazole assay. Conjugation of the fatty acid to heparosan ranging from 12 kDa to 40 kDa resulted in slower migration of the conjugate, as seen by 2% agarose gel electrophoresis with 1×TAE (Tris acetate EDTA; pH 8.3) buffer. Upon staining the gel with Stains-All (Sigma-Aldrich, St Louis, Mo.), the new compound had a similar color as the heparosan starting material, which is a bright blue (FIG. 3). Contrary to what was observed when employing dipalmitoyl phospholipid, this conjugate on its own did not aggregate into larger polydisperse structures under these conditions. This synthesis was performed employing heparosan oligomers as well as polymers with molecular masses ranging from the heparosan tetramer to polymer with a molecular mass of approximately 50 kDa.

### II. Synthesis of HEP4-Palmitoyl Derivative (FIG. 2A)

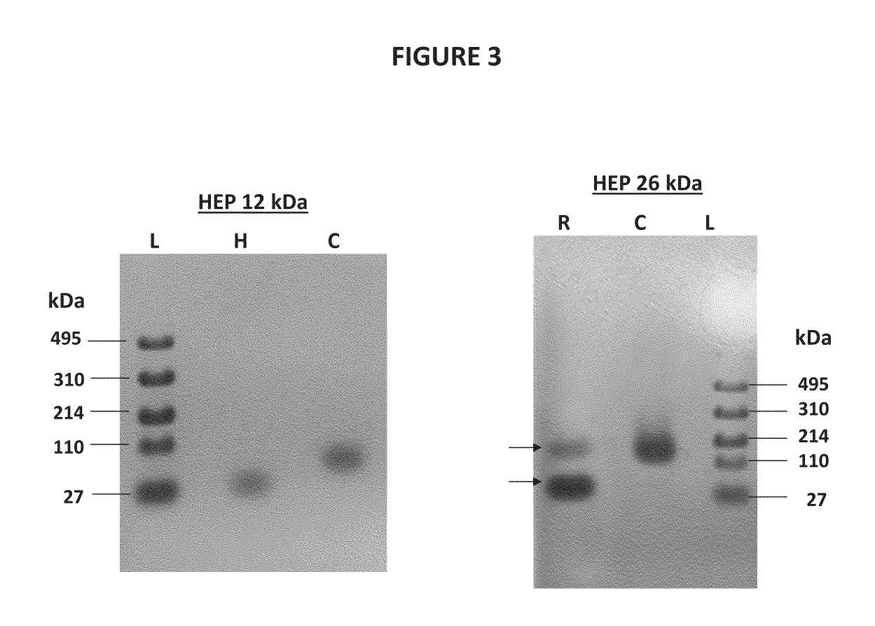

In one embodiment, heparosan oligosaccharides (here HEP-tetrasaccharide or HEP4) containing one primary amine linker at the reducing end of each oligosaccharide chain (synthesized by chemoenzymatic polymerization as described herein above) was reacted with 50-300 molar equivalents of N-Succinimidyl palmitate in a 96% aqueous dimethylsulfoxide solution containing 40 mM sodium phosphate, pH 7.0, at ambient temperature for 16 hours. However, N-succinimidyl esters of other fatty acids, as well as different solvent systems, solvent ratios, buffers, pH ranges, temperatures, and reaction times can equally be employed to synthesize conjugates between heparosan and fatty acids; the selection of the different variables is well within the skill of one of ordinary skill in the art. The obtained reaction mixture was purified by strong anion exchange chromatography followed by chloroform extraction and a desalting step of size exclusion chromatography column; also, modification of this general protocol to some degree is well within the skill of those of ordinary skill in the art. The purified conjugate was quantified by carbazole assay. Conjugation of the fatty acid to heparosan tetrasaccharides was analyzed by thin layer chromatography (TLC, silica plates, running buffer 2:1:1 n-butanol/acetic acid/H₂O) and stained with 0.2% naphthoresorcinol (in 95% ethanol with 5% sulfuric acid, heat exposure 100° C./30 minutes) (FIG. 4). The calculated Rf (ratio to the front) values for HEP-tetramer and the conjugate (C) were 0.0825 and 0.44, respectively. That difference in mobility indicates the linkage between HEP-tetramer and the HM was successful (FIG. 4).

### III. Synthesis of HEP-Palmitoyl/Oleoyl Derivative (FIG. 2B)

In another embodiment, essentially monodisperse 26 kDa heparosan polymer containing one primary amine linker at the reducing end of each polymer chain was reacted at a concentration of 2.2 mg/mL with 55 molar equivalents of N-(succinimidyloxy-glutaryl)-L-α-phosphatidylethanolamine, 1-palmitoyl-2-oleoyl (NHS-NG-POPE, NOF Corporation, Tokyo, Japan) in a slightly cloudy solution of 80% aqueous dimethylsulfoxide containing 30 mM sodium phosphate, pH 7.0, at ambient temperature for 3 hours, followed by 16 hours at 4° C. Other reaction conditions, including, but not limited to, different polymer concentration and molar reagent ratios, as well as other aminophospholipids to synthesize analogous conjugates with different lipid structures, can equally be employed; the selection of the different variables is well within the skill of one of ordinary skill in the art.

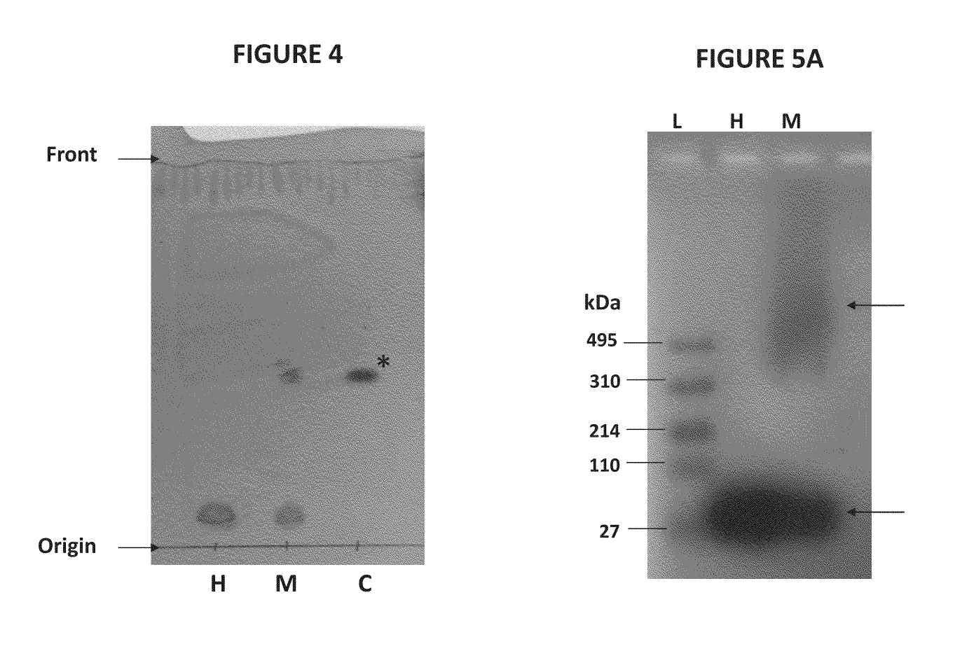

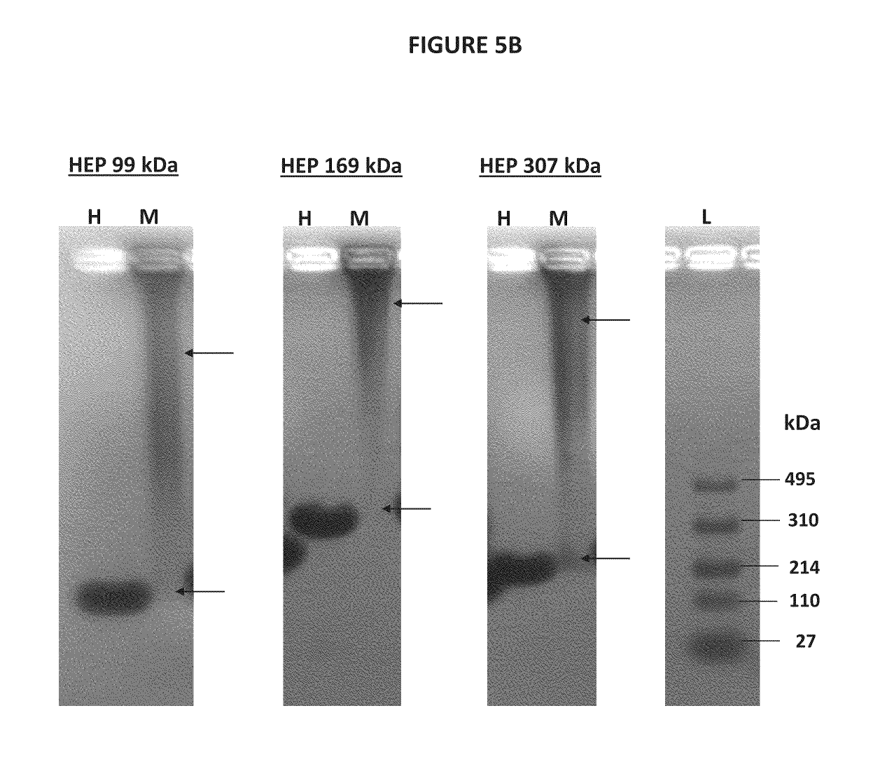

The reaction mixture was then semi-purified by precipitation with isopropyl alcohol (other water-miscible organic solvents such as acetone or ethanol can also be employed). Analysis by 1% agarose gel electrophoresis with lx TAE (Tris acetate EDTA; pH 8.3) buffer, followed by staining of the heparosan polymer with Stains-All (Sigma-Aldrich, St Louis, Mo.), showed that the formed heparosan-lipid conjugates mainly aggregated into larger polydisperse structures (micelles) under these conditions (FIG. 5). Anion exchange chromatography or other purification steps can be added to increase the purity of the final product.

### IV. Synthesis of HEP-Palmitoyl Derivative (Via Anhydride Route) (FIG. 2A)

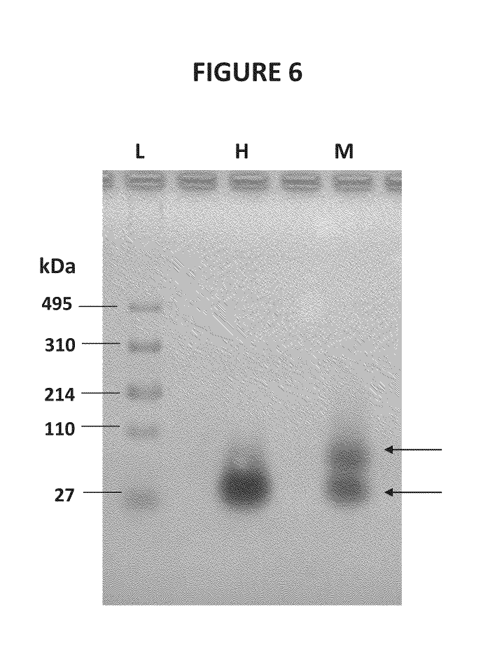

In one embodiment, quasi-monodisperse 29 kDa heparosan polymer containing one primary amine linker at the reducing end of each polymer was treated at a reaction concentration of 0.6 mg/mL with 740 molar equivalents palmitic anhydride and added solid sodium hydrogen carbonate at 6 mg/mL in a 20:10:10:1 (v:v:v:v) mixture of chloroform:methanol:dimethylsulfoxide:water at ambient temperature for 16 hours. However, other reaction conditions, including, but not limited to, other heparosan polymer sizes, anhydrides of other fatty acids, different solvents systems, temperatures, and molar ratios of reagents, can equally be employed to synthesize modified heparosan polymers containing fatty acids at their reducing end; the selection of the different variables is well within the skill of one of ordinary skill in the art. The crude reaction mixture was partly purified by isopropanol precipitation. Analysis by 1.5% agarose gel electrophoresis in 1×TAE (Tris acetate EDTA; pH 8.3) buffer, followed by staining of the heparosan polymer with Stains-All, showed that a heparosan-lipid conjugate had formed (FIG. 6). However, contrary to what was observed when employing dipalmitoyl phospholipid, this derivative with a single acyl tail did not aggregate on its own into larger aggregate structures under these conditions.

### V. Synthesis of HEP-Dipalmitoyl Derivative (Via Maleimide Route) (FIG. 2C)

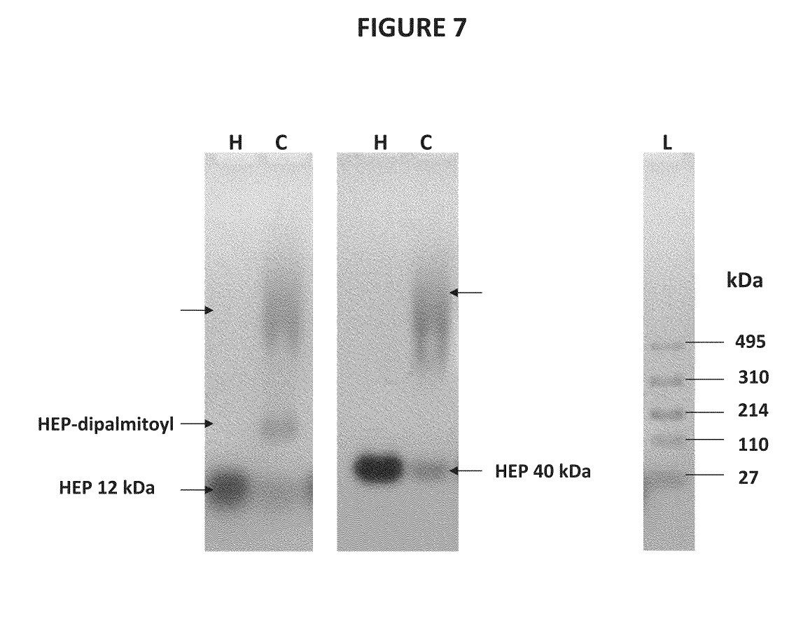

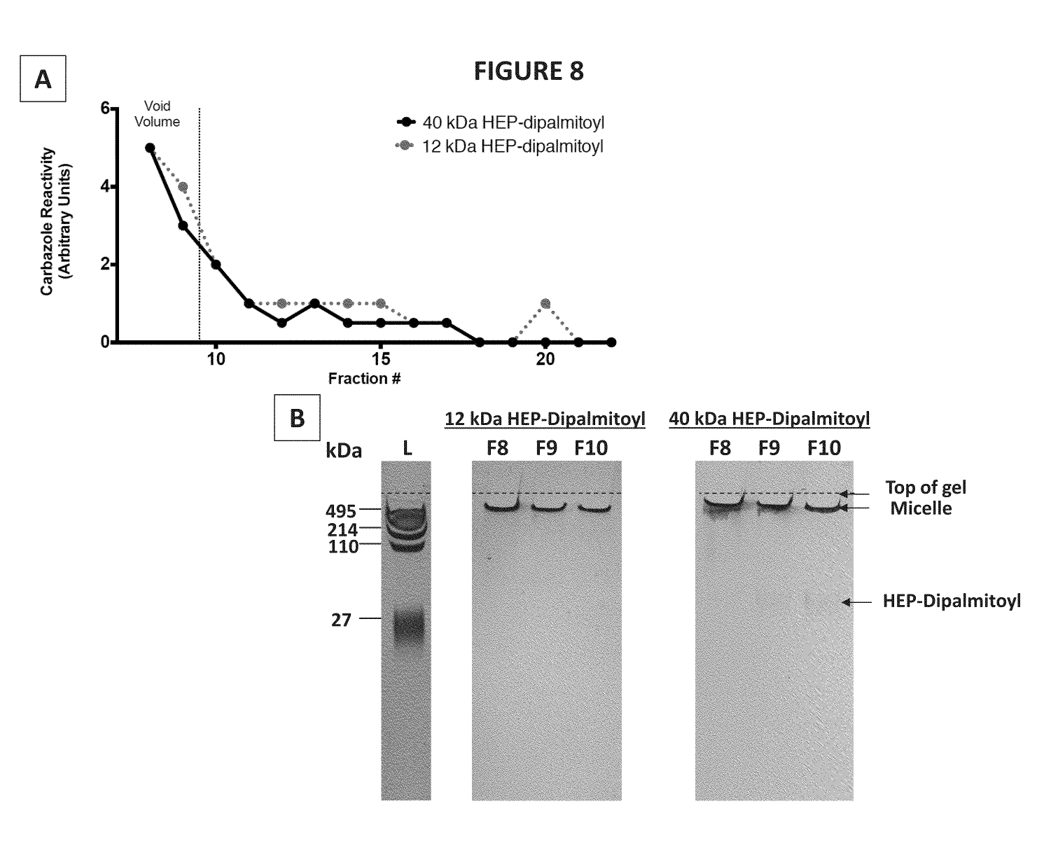

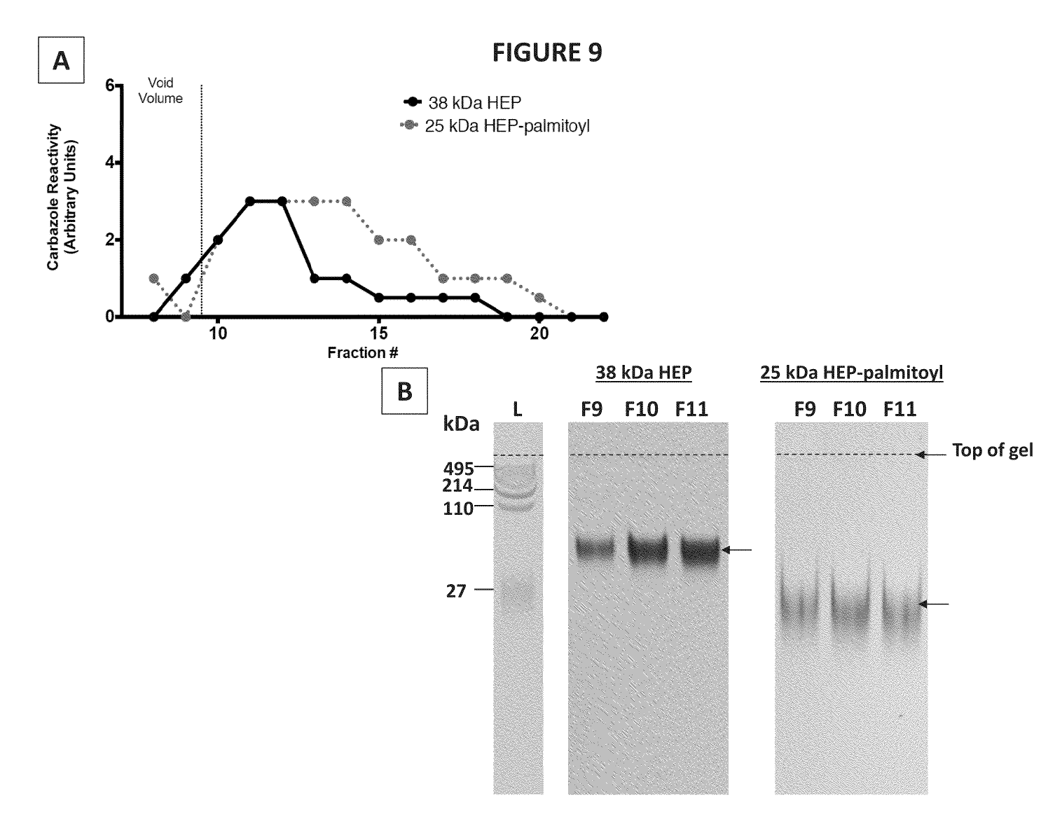

In one embodiment, essentially monodisperse heparosan containing one primary amine linker at the reducing end of each polymer chain was reacted at a reaction concentration of 22 mg/mL with 50 molar equivalents of heterobifunctional N-Sulfosuccinimidyl 4-maleimidobutyrate (sulfo-GMBS) in 100 mM sodium phosphate buffer, pH 7.0, for 1.5 hours at ambient temperature. Other reaction conditions, including, but not limited to, different polymer concentration and molar reagent ratios, can equally be employed; the selection of the different variables is well within the skill of one of ordinary skill in the art. The crude reaction mixture was purified by precipitation with isopropanol, and further purified by size-exclusion chromatography. Other purification methods, including, but not limited to, anion exchange chromatography and tangential flow filtration, can equally be employed, and the selection of the different methods for particular instances is well within the skill of one of ordinary skill in the art. The maleimide activated heparosan polymer containing a thiol-reactive maleimide group at the reducing end was subsequently reacted at a reaction concentration of 3 mg/mL with 10-30 molar equivalents of 1,2-dipalmitoyl-sn-glycero-3-phosphothioethanol in a 3:1:1 (v:v:v) mixture of dimethylsulfoxide:water:chloroform at 4° C. for 16 hours. Other reaction conditions, including, but not limited to, different polymer concentrations and molar reagent ratios, as well as other thiophospho-lipids, can equally be employed; the selection of the different variables is well within the skill of one of ordinary skill in the art. Next, unreacted lipids were removed by precipitation after addition of 2.5 volumes of 0.25 M sodium acetate, pH 7.0. Following centrifugation, the supernatant was collected, and the obtained pellet was purified by chloroform/water extraction. The aqueous phase was pooled with the supernatant collected earlier and analyzed by 1% agarose gel followed by Stains-All detection. This showed that the formed heparosan-lipid conjugates mainly aggregated into larger polydisperse structures (micelles) under these conditions (FIG. 7). Analysis by size exclusion of the HEP-dipalmitoyl (12 kDa or 40 kDa HEP polymer) conjugate confirmed the formation of micelles or aggregates, as most of the pre-purified HEP-dipalmitoyl eluted in the void volume (FIG. 8A), while HEP and HEP-palmitoyl elute later (FIG. 9). In addition, 6% PAGE analysis of the fraction showed that HEP-dipalmitoyl did not migrate through the gel due to size (FIG. 8B). Incubation in the presence of dipalmitic acid for 2 hours or 24 hours did not have any influence on the conversion yield or on the formation of micelles.

### VI. Synthesis of HEP-Thiocholesterol Derivative (Via Maleimide Route) (FIG. 2D)

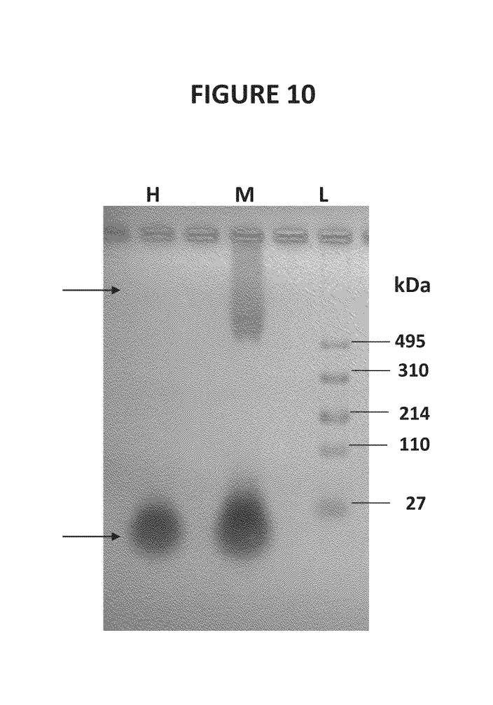

In one embodiment, essentially monodisperse 12.5 kDa heparosan polymer containing one primary amine linker at the reducing end of each polymer chain was treated at a reaction concentration of 22 mg/mL with 50 molar equivalents of heterobifunctional N-Sulfosuccinimidyl 4-maleimidobutyrate (sulfo-GMBS) in 100 mM sodium phosphate buffer, pH 7.0, for 1.5 hours at ambient temperature. However, other reaction conditions, including, but not limited to, different polymer concentration and molar reagent ratios, can equally be employed; the selection of the different variables is well within the skill of one of ordinary skill in the art. The crude reaction mixture was purified by precipitation with 1.5 volumes of isopropanol, and further purified by size-exclusion chromatography. However, other purification methods, including, but not limited to, anion exchange chromatography and tangential flow filtration, can equally be employed; the selection of the different methods for particular instances is well within the skill of one of ordinary skill in the art. The thus obtained activated heparosan polymer containing a thiol-reactive maleimide group at the reducing end was subsequently treated at a reaction concentration of 1.5 mg/mL with 290 molar equivalents of thiocholesterol in a 30:15:5:1 (v:v:v:v) mixture of chloroform:dimethylsulfoxide:methanol:water at ambient temperature for 16 hours. The crude reaction mixture was partly purified by isopropanol precipitation, and the obtained residue was dissolved in water. Analysis by 1.5% agarose gel electrophoresis with lx TAE (Tris acetate EDTA; pH 8.3) buffer, followed by staining of the heparosan polymer with Stains-All, showed that the formed heparosan-lipid conjugates self-aggregated into larger polydisperse structures (micelles) under these conditions (FIG. 10).

### VII. Synthesis of HEP-Distearoyl Derivative (Via Squarate Route) (FIG. 2E)

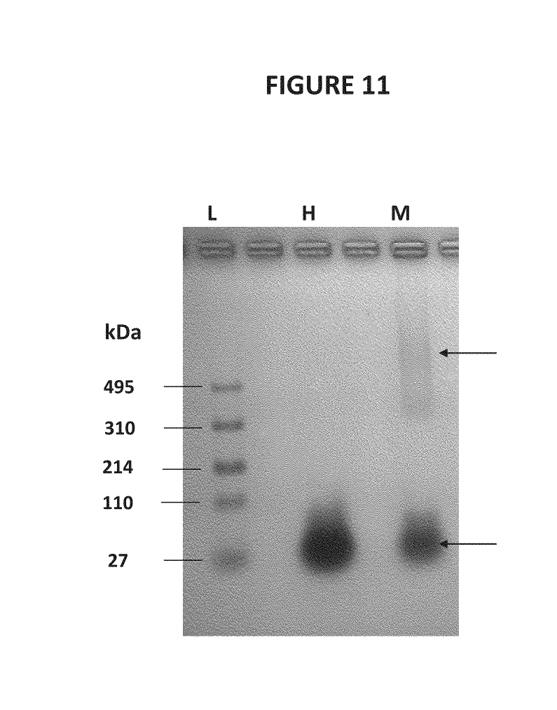

In one embodiment, essentially monodisperse 26 kDa heparosan polymer containing one primary amine linker at the reducing end of each polymer chain was activated by reaction at a concentration of 18 mg/mL with 130 molar equivalents of 3,4-diethoxy-3-cyclobutene-1,2-dione in 17% ethanol, containing 75 mM sodium phosphate, pH 7 to 7.5, at room temperature. The pH of the reaction mixture was monitored, and sodium hydrogen carbonate was added as needed to keep the pH between pH 7 to 7.5. After 2 hours, the reaction mixture was filtered and purified by size exclusion chromatography. The activated heparosan polymer absorbs strongly at 280 nm, while heparosan alone does not absorb UV at this wavelength. Other heparosan polymer sizes and purification methods, including, but not limited to, anion exchange chromatography, can be used as well. The activated heparosan polymer was subsequently reacted at a reaction concentration of 0.3 mg/mL with 300 molar equivalents of 1,2-distearoyl-syn-glycero-3-phosphoethanolamine in a 12:12:6:1 (v:v:v:v) mixture of chloroform:methanol:dimethylsulfoxide:100 mM sodium phosphate, pH 7.0 to 8.5, at room temperature for 16 hours. The pH and buffer for this HM-coupling step is typically more alkaline than the initial HEP activation; the selection of the different variables is well within the skill of one of ordinary skill in the art. The polymer was precipitated by addition of isopropanol, dried in vacuo, and dissolved in water. Analysis by 1% agarose gel electrophoresis in 1×TAE (Tris acetate EDTA; pH 8.3) buffer, followed by staining of the heparosan polymer with Stains-All (Sigma-Aldrich, St Louis, Mo.), showed that the formed heparosan-lipid conjugates aggregated into larger polydisperse structures (micelles) under these conditions (FIG. 11).

### VIII. Synthesis of HEP-Fluorolipid Derivative (FIG. 2F)

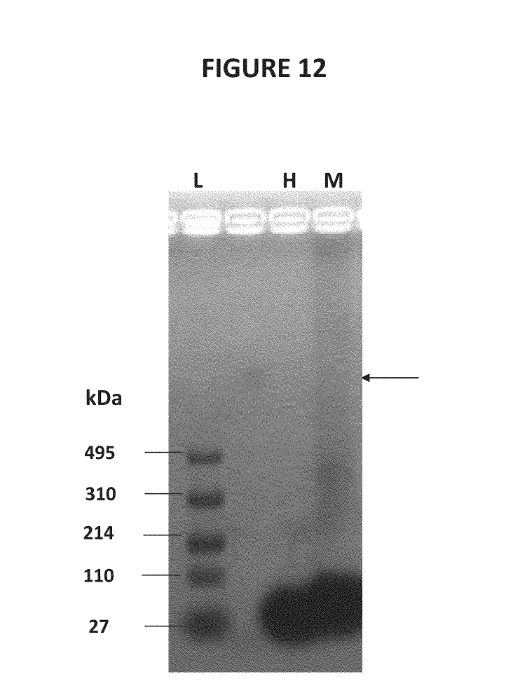

In one embodiment, essentially monodisperse 29 kDa heparosan containing one primary amine linker at the reducing end of each polymer was reacted with 55 molar equivalents of heterobifunctional N-succinimidyl S-Acetylthioglycolate (SATA, TCI America, Portland, Oreg.) in 100 mM sodium phosphate buffer, pH 7.0, containing 15 vol % dimethylsulfoxide for 1.5 hours at ambient temperature. However, other reaction conditions, including, but not limited to, different polymer concentration and molar reagent ratios, can equally be employed; the selection of the different variables is well within the skill of one of ordinary skill in the art. The crude reaction mixture was purified by isopropanol precipitation and further purified by tangential flow filtration (TFF). However, other purification methods, including, but not limited to, anion exchange chromatography and size-exclusion chromatography, can equally be employed; the selection of the different methods for use in particular instances is well within the skill of one of ordinary skill in the art. The thus obtained activated heparosan polymer containing a thioacetyl group at the reducing end was subsequently treated at a reaction concentration of 1.44 mg/mL with 50 mM hydroxylamine and 5 mM EDTA in 50 mM sodium phosphate, pH 7.0, for 20 minutes at ambient temperature, after which time the solution was added to 2300 molar equivalents of N-(4,4,5,5,6,6,7,7,8,8,9,9,10,10,11,11,11-heptadecafluoroundecyl)maleimide in a 1:1 solution of dimethylsulfoxide and dimethylformamide at 1.6 mg/mL at ambient temperature. The reaction was agitated for 16 hours. However, other reaction conditions, including, but not limited to, different polymer concentration, solvent systems, and molar reagent ratios, as well as other maleimide reagents containing different fluoroalkyl side chain length, can equally be employed to synthesize analogous conjugates; the selection of the different variables is well within the skill of one of ordinary skill in the art. The polymer was partially purified by precipitation with isopropanol. Analysis by 1% agarose gel electrophoresis in 1×TAE (Tris acetate EDTA; pH 8.3) buffer followed by staining of the heparosan polymer with Stains-All showed that the formed heparosan-lipid conjugates aggregated into larger polydisperse structures (micelles; FIG. 12).

### Example 3

### Preparation of Liposomes

Liposomes with different lipid compositions were produced by several procedures as mentioned in Table 4 and elsewhere. In addition, some of these preparations also contained lumen-entrapped small molecular weight (MW) molecules of several classes including but not limited to: carboxyfluorescein-dye (Sigma-Aldrich, St Louis, Mo.), an antibiotic such as tetracycline (Sigma-Aldrich, St Louis, Mo.), or a chemotherapy agent such as doxorubicin (Fluka Analytical, Switzerland). Free small MW reagents were removed by gel filtration or centrifugation before use. In addition, virtually any hydrophilic water-soluble molecule can be entrapped in liposomes, including polypeptides and polynucleotides. Liposome of preparation #5, non-PEGylated DOXOSOME® (Encapsula NanoSciences LLC, Brentwood, Tenn.), was preloaded with 2 mg/ml doxorubicin.

TABLE 4
Liposome Types Tested

| Prep | | Preparation | |
|---|---|---|---|
| # | Lipid Composition^{a} (mol %) | Method ^{b} | Note |
| 1 | PC (10%), CL (10%), PG | D/S/E | bacterial-like |
| | (60%), PE (10%), and PE (10%) | | |
| 2 | PC (46.5%), PE (28.4%), PI | D/FT/E | mitochondrial- |
| | (8.9%), PS (8.9%), and | | like |
| | CA (7.3%) | | |
| 3 | synthetic PC (45%) and | D/E | synthetic |
| | Chol (55%) | | |
| 4 | hydrogenated soybean PC | EE/S | DOXIL ®*-like |
| | (59%) and Chol (41%) | | |
| 5 | Hydrogenated soybean PC | HPE | Commercially |
| | (60%) and Chol (40%) | | available |
| | | | DOXOSOME ®* |

^{a}standard abbreviations of lipidomics in Table 5;
Chol = cholesterol
^{b} dry film, D; sonication, S; extrusion through filters, E; extrusion into ethanol, EE; freeze-thaw, FT; high pressure extrusion through filters, HPE.
*PEG-coated liposomes with entrapped doxorubicin; Janssen Products, LP; Titusville, NJ
**doxorubicin liposomes (Encapsula Nanosciences LLC, Brentwood, TN)

TABLE 5
Standard Abbreviations of Lipidomics

| Class | Abbreviation |
|---|---|
| Glycerophosphocholines | PC (LPC for lyso species) |
| Glycerophosphoethanolamines | PE (LPE for lyso species) |
| Glycerophosphoserines | PS (LPS for lyso species) |
| Glycerophosphoglycerols | PG (LPG for lyso species) |
| Glycerophosphates | PA (LPA for lyso species) |
| Glycerophosphoinositols | PI (LPI for lyso species) |
| Glycerophosphoinositol monophosphates | PIP |
| Glycerophosphoinositol bis-phosphates | PIP2 |
| Glycerophosphoinositol tris-phosphates | PIP3 |
| Glycerophosphoglycerophosphoglycerols | CL |
| (Cardiolipins) | |
| Glycerophosphoglycerophosphates | PGP |
| Glyceropyrophosphates | PPA |
| CDP-glycerols | CDP-DG |
| Glycosylglycerophospholipids | [glycan]-GP |
| Glycerophosphoinositolglycans | [glycan]-PI |
| Glycerophosphonocholines | PnC |
| Glycerophosphonoethanolamines | PnE |

To assess HEP-HM incorporation into liposomes to form a HEPosome, various standard separation processes were employed to separate the molecules in the free soluble aqueous state from the lipid aggregate/liposome-bound fractions; these include (but are not limited to): (a) sucrose gradient flotation (25-30% sucrose pads with 150,000×g for 1.5 hour centrifugation); (b) Percoll flotation (85-95% Percoll with 20,000×g centrifugation for 45 minutes); or (c) centrifugation sedimentation (20,000-100,000×g for 10-90 minutes).

For methods (a) and (b), the liposomes (with low relative density) rise to the top or ‘float.’ For the latter method (c), the liposomes are found in pellets at the bottom and were washed with buffer before harvesting. These methods, as well as gel filtration chromatography, can also be used to remove unincorporated drug from liposomes with entrapped drugs. In one embodiment, the incorporation of HEP-HM into the liposome is a ‘post-modification’ of the liposomes. Once the liposomes were synthesized, HEP-HMs were added to the liposome suspension and were therefore incorporated only on the outer lipid layer. This method is described below through examples.

In one embodiment, the alternative method to the ‘post-modification’ addition of HEP-HM to preformed liposomes described below, the HEP-HM can also be present at the very beginning of the liposome preparation. For example, dry films of lipids and HEP-HM can be hydrated in aqueous buffers to make the HEPosome. In another embodiment, the HEP-HM could be added at the initial phase of extrusion of lipids in a solvent (e.g., ethanol) into an aqueous solution. However, these two types of formulations where the HEP-HM is present at the beginning of the liposome production process may have some portion of the HEP molecules facing the lumen or interior of the liposome, where they will not be effective in shielding the surface of the liposome when in the body. In addition, the HEP polymer would take up some of the limited finite space in the lumen where the drug cargo will be present, thus reducing the load of therapeutic/potential therapeutic agent in the liposome.

### Example 4

### Addition of HEP-HM to Liposomes Via ‘Post-Modification’ Route

Incorporation of HEP-palmitoyl or HEP-dipalmitoyl to a liposome in a ‘post-modification’ route involves adding the HEP-HM to a pre-formed liposome, and this method can be modified to some degree, as is well within the skill of one of ordinary skill in the art. Simple addition of the HEP-HM reagent (e.g, HEP-palmitoyl, etc.; at various ratios to effect surface coverage) to the liposome aqueous solution at neutral pH (typically in saline buffer pH 7-7.5) under mild swirling conditions resulted in incorporation of HEP-HM into the liposomes within 15-30 minutes at room temperature. Other buffers, incubation temperature and times, and different pH, or molar ratios may substitute, as is well within the skill of a person of ordinary skill in the art to determine. The use of overnight to 1 week incubation periods did not lead to a major increase in the incorporation of HEP-HM. On the other hand, in parallel experiments, heparosan without a HM (i.e., a polar, hydrophilic polysaccharide reagent alone) did not incorporate into the liposomes.

The level of heparosan chains anchored onto the liposomes was significant, as observed by the electrophoretic behavior of the liposomes. In the early stage of electrophoresis, it was noticed by visual observation that liposomes without HEP occupied the entire volume of a well of an agarose gel when subjected to an electric field (approximately 30-80 Volts). However, incorporation of HEP-palmitoyl into the identical liposomes allowed the liposomes to migrate towards the positive side of the well, which is consistent with their possessing the polyanionic (highly negatively charged) heparosan molecule. Over time, the heparosan-lipid appeared to be ‘ripped’ out of the liposome and started running in the gel, where it was observed as a smearing or trailing band of staining (i.e., not a discrete band, as seen in parallel samples of a given HEP-HM without any liposomes).

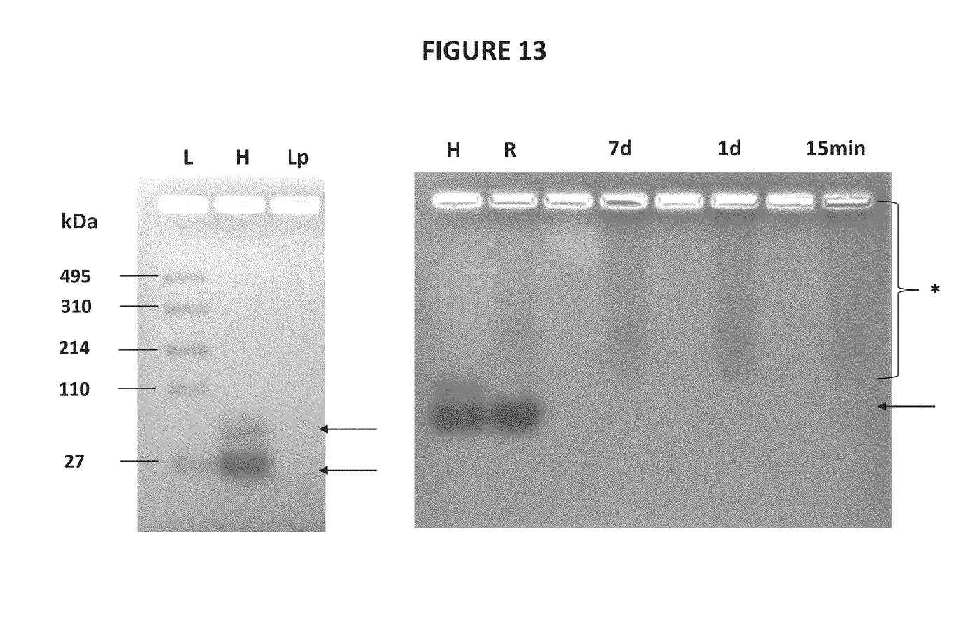

The liposomes coated with HEP were stable for at least 1-7 days at 4° C. in neutral saline buffers. In addition, the entrapped dye stayed within the liposomes, and thus the liposome was not excessively leaky, as compared to the liposome controls without any HEP-HMs. Independent of the lipid composition of the liposomes, the incorporation of HEP-HM into the liposomes was successful, as confirmed by agarose gel analysis (FIGS. 13 to 16).

In FIG. 13 (left gel), the ‘H’ lane was loaded with a semi-purified reaction mixture of 28 kDa HEP-amine and NHS-palmitate; the upper band present in this lane is the HEP-palmitoyl, while the lower band is unreacted HEP-amine (approximately 20:80 ratio of these two species, respectively). The ‘Lp’ lane contains liposomes which did not stain. The incorporation of HEP-palmitoyl into liposomes (prep #3) was tested in 15 minutes, 1 day, or 7 day incubation periods at room temperature (approximately 20-25° C.) followed by centrifugation to collect the liposome fraction at these times. These pellets containing the liposomes were analyzed (FIG. 13; right gel). A light to moderate increase in the incorporation of HEP-HM into liposomes was observed between a 15-minute incubation and a 1-day incubation; however, no increase was observed after incubation for 7 days. In the mixture of HEP plus liposomes (R), the slower mobility HEP-palmitoyl associates with the liposomes (Prep #3 loaded with carboxyfluorescein dye, a ‘drug proxy’ for doxorubicin, antibiotics, steroid, etc.); in fact, this hydrophobic heparosan derivative polymer smeared during gel analysis, as it was apparently pulled off (by the electromotive force) of the liposomes trapped in the well at the top of the gel.

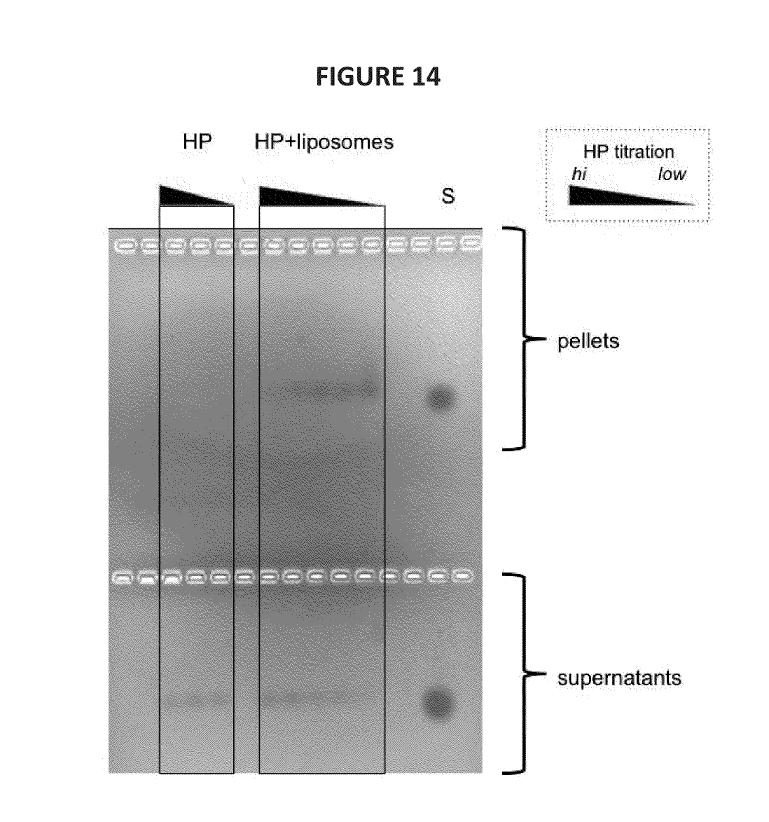

A titration of 28 kDa HEP-palmitoyl (HP; triangle indicates the direction of concentration) with a fixed amount of liposomes (Prep #3) was performed (FIG. 14). After incubation, centrifugation was used to separate the free HP from the bound HP, and the presence of the sugar was then detected by gel/Stains-All analysis. The gel was loaded so that equal mass amounts of HP (based on the initial reaction) were loaded on the gel (i.e., less volume of the more concentrated HP reaction samples was loaded per lane and vice versa). The upper panel lanes contain the pellets (liposome-bound HP), while the lower panel lanes contain the supernatants (free HP). The ‘HP+liposomes’ (on right) show the presence of liposomes with heparosan in the pellet; conversely, the negative controls without liposomes (′HP′ on the left) show the staining of heparosan in the supernatants, not the pellets. A saturation effect is seen; at low concentrations, most HP is with the liposome, but at higher levels, there is excess HP, which is seen more in the supernatants. This observation is because the liposomes only have so much HP-binding capacity due to a finite surface area. Also, due to the normalization of equal input HP loading per lane, there is not ever-increasing amounts of HP in the pellet in an effort to avoid over-loading of the gel for better quantification (S=HEP-palmitoyl standard; 1 or 2 micrograms, top and bottom panels, respectively).

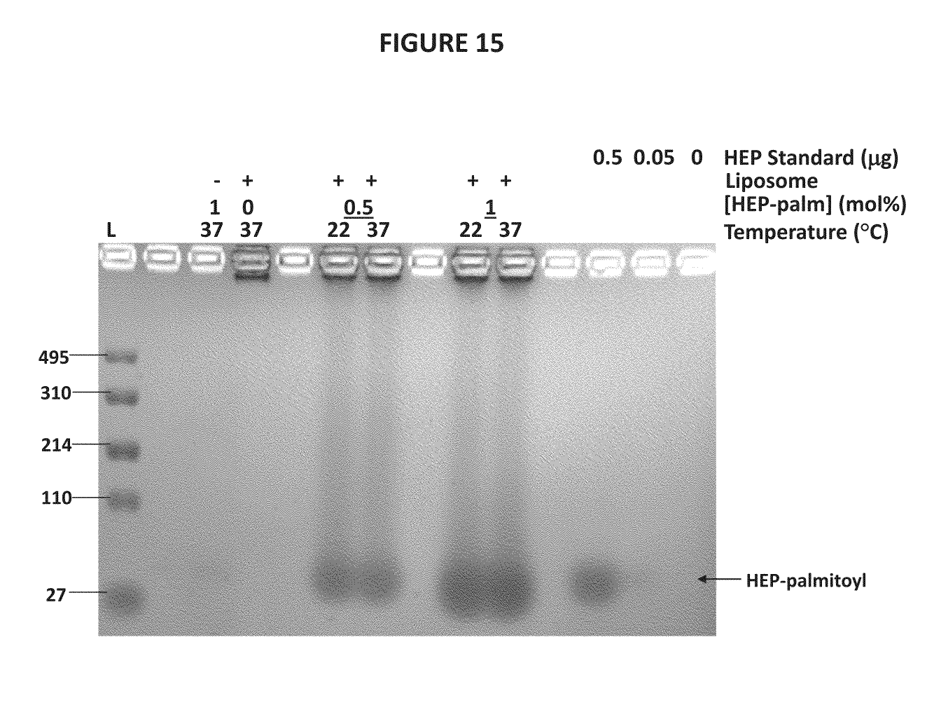

The effect of the incubation temperature and the molar ratio of HEP-palmitoyl was also evaluated for the incorporation of this HEP-HM into doxorubicin-loaded liposomes (prep #5), as measured by centrifugation and gel analysis (FIG. 15). It was found that incubation temperature in the range of 22 to 37° C. did not have a significant effect on the incorporation of HEP-palmitoyl into the liposome. More HEP-palmitoyl was incorporated into liposomes when this reagent was added at 1 molar % in comparison to 0.5 molar %.

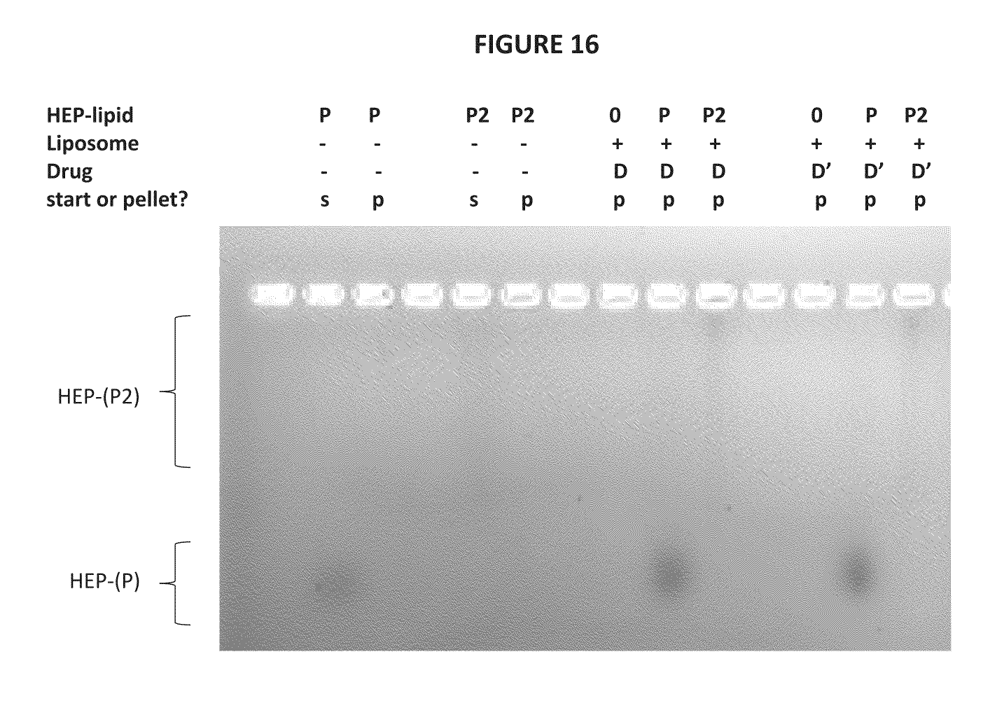

Other liposomes with entrapped molecules were also tested (FIG. 16). Either a solution of 28 kDa HEP-palmitoyl (P), 13 kDa HEP-dipalmitoyl (P2), or water alone (0; a negative control) were mixed with drug-loaded liposomes (prep #4 with either entrapped doxorubicin ‘D’ or tetracycline ‘T’ as noted; drug-saturated ethanol solution was mixed with lipids in ethanol, then extruded into 10 volumes of vigorously mixed saline). After a 30-minute incubation, the heparosan/liposome mixtures were diluted with saline buffer, and centrifugation was used to separate the free HEP-lipid from the bound HEP-lipid in liposomes. Samples run on the gel were either the starting mixture (′start′ which reflects the total input materials) or the washed pellet (‘p’ which reflects the liposomally-associated materials). The presence of the sugar was detected by gel/Stains-All analysis. The presence of the drugs was detected by visible color/fluorescence (red/red for D and yellow/green for T). The HEP-HM and drug fractionated with the liposomes in the pellets, as seen by the bands in the ‘p’ lanes in the two sets of lanes on the right half of the gel.

I. Toxin Challenge of Dye-Loaded Liposomes with or without HEP-HM

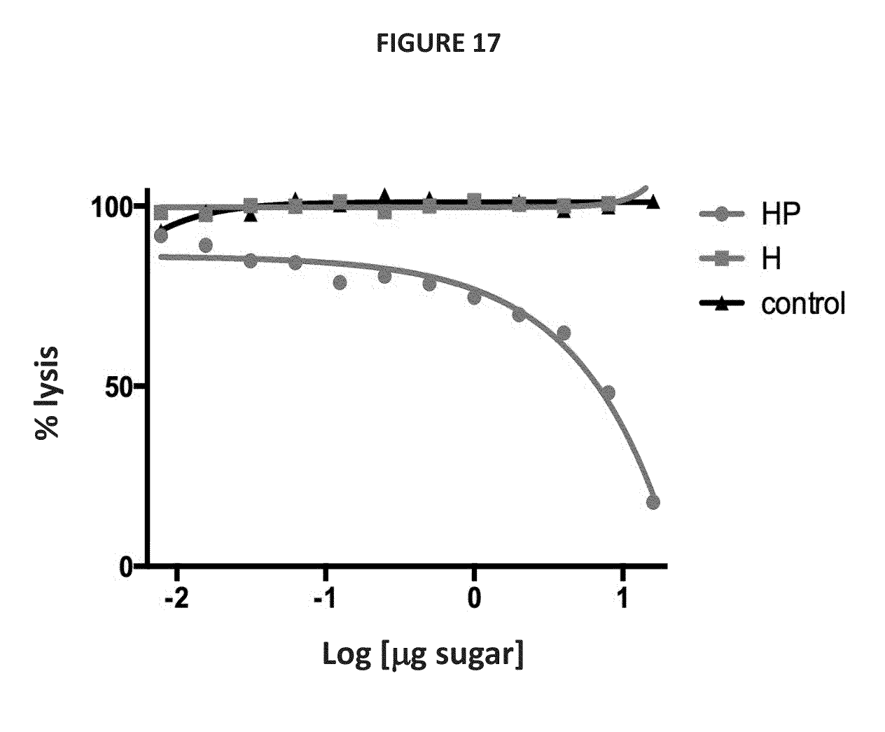

In order to assess the robustness and the physical nature of the HEP coating on a liposome, the liposomes were challenged with Perfringolysin O toxin (from Clostridium perfringens); this protein will destroy normal uncoated liposomes and normal cells upon contact with the cholesterol present in the lipid bilayer. A titration of a solution of 28 kDa HEP-palmitoyl (HP, circles), 28 kDa HEP (H, squares; the starting material without a hydrophobic group thus a negative control), or water (another negative control, triangles) was mixed with dye-loaded liposomes (prep #3 with entrapped carboxyfluorescein) in a 96-well format. After a 30-minute incubation at 37° C., 45 nanograms of Perfringolysin O toxin (3 times the EC₅₀ dose used per well so significant lysis would occur during the assay without any inhibitor) was added to each well and incubated again for 1 hour before reading the fluorescent signal (when liposomes lyse, the quenching caused by the entrapped dye at high concentration is relieved; thus, higher levels of fluorescence is observed). Heparosan-palmitoyl protects the liposomes from lysis, but the heparosan alone (which does not incorporate into liposomes) and the water control do not. Thus, the HEPosome is protected by virtue of its heparosan coating. This model system with bacterial pore-forming toxins is similar biophysically to the attacks mediated by the defenses of the mammalian patient (e.g., the complement system). The results showed that the HEP coating protects the liposome surface against the toxin (FIG. 17).

### II. Efficacy of HEPosomes in a Mammalian Disease Model

The efficacy of HEP-coated (12 kDa-palmitoyl derivatized polymer) Doxorubicin-entrapped liposomes (a type of HEPosome) to inhibit tumors was determined using the 4T1 murine mammary tumor model. The tumor growth and metastatic spread of 4T1 cells in BALB/c mice very closely mimics stage IV human breast cancer. The 4T1 tumor bearing Balb/c mice received HEP-coated DOXOSOME®s (HLD), PEGylated DOXOSOME®s (PLD), naked (only lipids and cholesterol; NLD) DOXOSOME®s, or saline vehicle by the intravenous (IV) route via tail vein. The NLD and PLD liposomes were from Encapsula NanoSciences LLC (Brentwood, Tenn.). The NLD (the naked, non-PEGylated DOXOSOME®s) were coated with a HEP-HM (12 kDa-HEP-palmitoyl) at a concentration of 1 molar % for 2 hours at room temperature before injection.

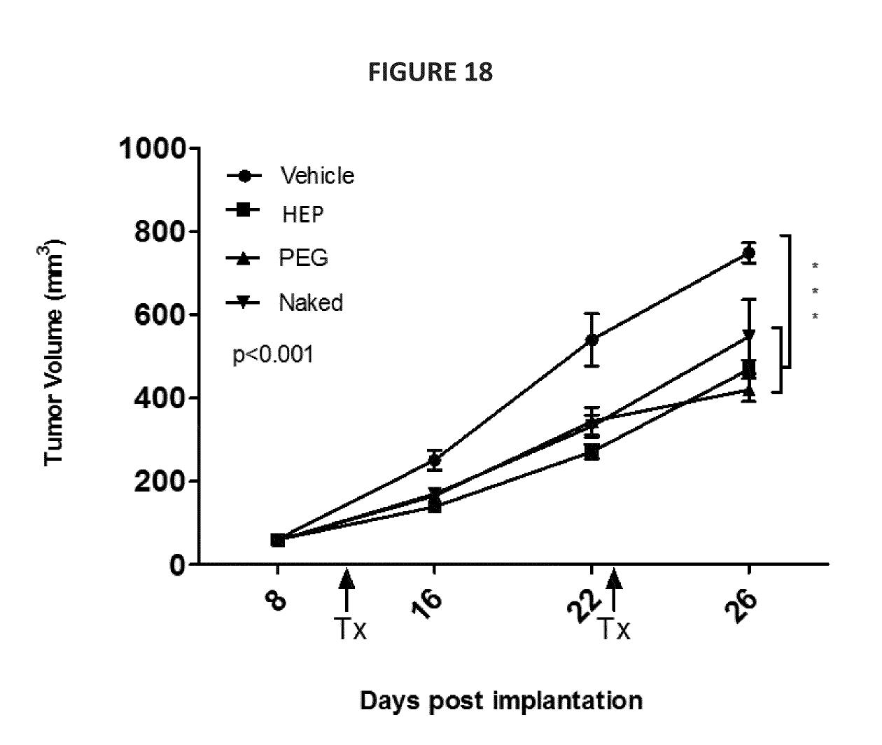

Ten days after injections of 1×10⁵ 4 T1 cells (in the fourth mammary fat pad), the Balb/c mice with confirmed palpable tumors (Experlmmune, Austin, Tex.) were placed into the following treatment groups (each n=10): (i) vehicle control (saline, 100 μl); (ii) PLD 6 mg/kg (based on doxorubicin content); (iii) HLD 6 mg/kg; and (iv) NLD 6 mg/kg. Tumors were measured by caliper to give a tumor volume (FIG. 18). The efficacy study indicates a comparable response between the three DOXOSOME® formulations, with a trend of improved efficacy arising in the HEP-coated liposome treated group compared to the naked liposome group.

### Example 5

### Micelle Formation and Drug Loading

Many current drugs and drug candidates are small molecules with poor water solubility and low permeability; thus, significant classes of drug/drug candidates require delivery systems. In one embodiment, HEP-dipalmitoyl and/or HEP-palmitoyl/oleoyl and other HEP-HM derivatives, with HEP polymers of different molecular weights, have been shown herein to be capable of forming stable micelles; these multimolecular aggregates can be purified through size exclusion chromatography in aqueous solution and possess much higher apparent molecular weights than expected for the HEP-HM monomers. Another indication of these micellar aggregates is their mobility in electrophoretic gels; the micelles run much slower than the original monomers (FIGS. 9 and 10).

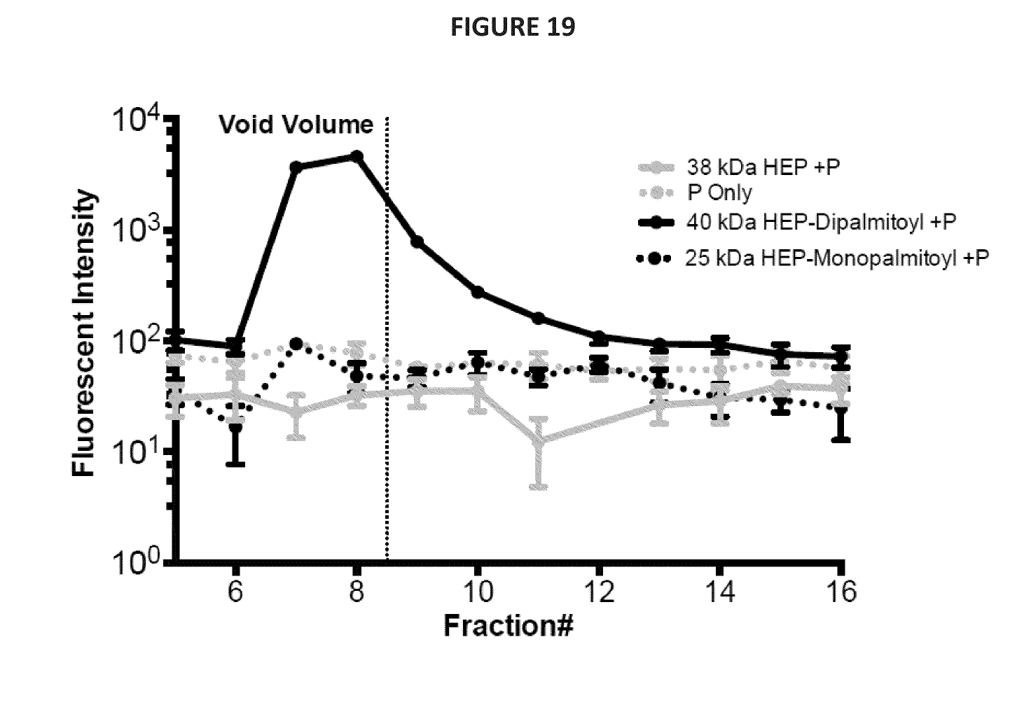

In one non-limiting example, 40 kDa HEP-dipalmitoyl (150 μg on a sugar basis) was incubated with primuline (Direct Yellow 59 or Primuline Yellow; 2.5 μg; Sigma-Aldrich, St. Louis, Mo.), a relatively hydrophobic dye used in the field to track lipids. Primuline is used here as a ‘proxy’ for a drug to show the capacity of HEP-HM to form micelles exhibiting drug cargo-carrying activity. As control, 25 kDa HEP-palmitoyl, 38 kDa HEP (with no hydrophobic anchor), and primuline (all at similar concentrations as above) alone were analyzed by size exclusion chromatography on SEPHAROSE 6B® (GE Healthcare Bio-Sciences, Pittsburgh, Pa.). On this resin (fractionation range [Mr] approximately 1×10⁴-4×10⁶; Exclusion Limit >4×10⁶), HEP molecules that do not form a micelle will run in the partially included volume, while any aggregates (e.g., micelles) will run in the void or totally excluded fractions. The different fractions were analyzed for their primuline content by fluorescence measurement with excitation at 410 nm and emission at 550 nm (FIG. 19). The result of fluorescence in the void volume of the column indicates that HEP-dipalmitoyl formed micelles capable of carrying primuline in an aqueous solution; the parallel samples with either HEP-monopalmitoyl (no micelles under these conditions) or HEP alone (no hydrophobic moiety) did not exhibit this behavior. This finding demonstrates that the general HEP-micelle structure can be used as a drug delivery composition.

Thus, in accordance with the presently disclosed and/or claimed inventive concept(s), there have been provided compositions and methods that fully satisfy the objectives and advantages set forth hereinabove. Although the presently disclosed and/or claimed inventive concept(s) has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the presently disclosed and claimed inventive concept(s).

### REFERENCES

The following references, to the extent that they provide exemplary procedural or other details supplementary to those set forth herein, are specifically incorporated herein by reference.
Armstrong et al. Cancer (2007) 110(1):103-11.
Besheer et al. J Pharm Sci. (2009) 98(11):4420-8.
Capila et al. Angew Chem Int Ed Engl (2002) 41(3):391-412.
DeAngelis. Glycobiology (2002) 12(1):9R-16R.
Gaberc-Porekar et al. Curr Opin Drug Discov Devel. (2008) 11(2):242-50.
Ganson et al. Arthritis Res Ther. (2006) 8(1):R12. PMCID: 1526556.
Gregoriadis et al. Cell Mol Life Sci. (2000) 57(13-14):1964-9.
Hamad et al. Mol Immunol. (2008) 46(2):225-32.
Harris et al. Glycobiology (2008) 18(8):638-648.
Ishida et al. J Control Release (2007) 122(3):349-55.
Pasut et al. Journal of Controlled Release: official journal of the Controlled Release Society (2012) 161(2):461-472.
Pikas et al. The Journal of Biological Chemistry (1998) 273(30):18770-18777.
Pisal et al. Journal of Pharmaceutical Sciences (2010) 99(6):2557-2575.
Sismey-Ragatz et al. The Journal of Biological Chemistry (2007) 282(39):28321-28327.
Sugahara et al. IUBMB Life (2002) 54(4):163-175.
Sundy et al. Arthritis Rheum (2007) 56(3):1021-8.
Verhoef et al. Drug Discovery Today (2014) September 7. pii: S1359-6446(14)00342-0. doi: 10.1016/j.drudis.2014.08.015. [Epub ahead of print]
Veronese. Biomaterials (2001) 22(5):405-17.
Wagner et al. J. of Drug Delivery (2011)—Volume 2011, Article ID 591325, 9 pages, doi:10.1155/2011/591325

What is claimed is:

1. A drug delivery composition, comprising:
a multimolecular assembly formed of a plurality of components, at least a portion of the plurality of components comprising at least one hydrophobic moiety, wherein the plurality of components aggregate together via hydrophobic interactions to form the multimolecular assembly;
at least one heparosan polymer having a hydrophobic moiety-containing component attached thereto, wherein the hydrophobic moiety is incorporated into the multimolecular assembly whereby the at least one heparosan polymer is attached to a surface of the multimolecular assembly, and wherein the heparosan polymer is characterized as being substantially non-antigenic, substantially non-immunogenic, and substantially biologically inert within extracellular compartments of a mammalian patient, being stable in the mammalian bloodstream, and being degraded intracellularly in the mammalian patient; and
at least one therapeutic agent and/or potential therapeutic agent entrapped, carried, and/or bound in the multimolecular assembly.

2. The drug delivery composition of claim 1, wherein the at least one therapeutic/potential therapeutic agent is selected from the group consisting of a chemotherapy agent, an antineoplastic agent, a steroid, an antibiotic, an anti-inflammatory agent, an agent that has an action on a central nervous system of the mammalian patient, an antihistaminic, an antiallergic agent, an antipyretic, a respiratory agent, an antimicrobial agent, an antihypertensive agent, a calcium antagonist, an antipsychotic, an agent for Parkinson's disease, a vitamin, an antitumor agent, a cholinergic agonist, a mydriatic, an antidepressant agent, an antidiabetic drug, an anorectic agent, an antimalarial agent, a polypeptide therapeutic, a cytokine, a hormone, an enzyme, an antibody, an antibody fragment, an antiulcerative agent, an anticancer agent, a vaccine antigen, a polynucleotide, a nutrient, a small molecule, and combinations thereof.

3. The drug delivery composition of claim 1, wherein the multimolecular assembly is a monolayer.

4. The drug delivery composition of claim 3, wherein the multimolecular assembly is a micelle.

5. The drug delivery composition of claim 4, wherein:
(a) the micelle has a spherical, ellipsoid, and/or cylindrical shape; and/or
(b) the micelle is a polymeric micelle.

6. The drug delivery composition of claim 1, wherein the multimolecular assembly is a bilayer.

7. The drug delivery composition of claim 1, wherein the multimolecular assembly is a vesicle.

8. The drug delivery composition of claim 7, wherein the vesicle is a liposome.

9. The drug delivery composition of claim 7, wherein the vesicle is selected from the group consisting of a multilamellar vesicle (MLV), a small unilamellar vesicle (SUV), a large unilamellar vesicle (LUV), a cochleate vesicle, and combinations thereof.

10. The drug delivery composition of claim 1, wherein the multimolecular assembly is an aggregate of micelle(s) and/or vesicle(s).

11. The drug delivery composition of claim 1, wherein each of the plurality of components is selected from the group consisting of a lipid, a fatty acid, an alkyl chain, a hydrocarbon-rich group, a fluorocarbon-rich group, a sterol, and combinations thereof.

12. The drug delivery composition of claim 1, wherein at least one of the plurality of components is selected from the group consisting of a palmitoyl chain, an oleoyl chain, a stearoyl chain, an alkyl chain with 8 to 20 carbons and zero to multiple unsaturated bonds, a chain with 2 to 20 carbons with multiple fluorine atoms, and combinations thereof.

13. The drug delivery composition of claim 1, wherein the heparosan polymer has a mass in a range of from about 600 Da to about 300 kDa.

14. The drug delivery composition of claim 1, wherein the multimolecular assembly comprises a plurality of heparosan polymers/hydrophobic moieties incorporated therein, and wherein the plurality of heparosan polymers is polydisperse in size.

15. The drug delivery composition of claim 1, wherein the multimolecular assembly comprises a plurality of heparosan polymers/hydrophobic moieties incorporated therein, and wherein the plurality of heparosan polymers is substantially monodisperse in size, and wherein at least one of:
(a) the substantially monodisperse heparosan polymers have a molecular weight in a range of from about 3.5 kDa to about 300 kDa and a polydispersity value in a range of from about 1.0 to about 1.1;
(b) the substantially monodisperse heparosan polymers have a molecular weight in a range of from about 0.5 MDa to about 300 kDa and a polydispersity value in a range of from about 1.0 to about 1.5; and
(c) the substantially monodisperse heparosan polymers have a molecular weight in a range of from about 0.5 MDa to about 300 kDa and a polydispersity value in a range of from about 1.0 to about 1.2.

16. The drug delivery composition of claim 1, wherein the heparosan polymer is a linear chain.

17. The drug delivery composition of claim 1, wherein the heparosan polymer has a branched geometry.

18. The drug delivery composition of claim 1, wherein at least one of:
(a) the drug delivery composition exhibits increased retention in blood and/or lymphatic circulation of a mammalian patient when compared to therapeutic/potential therapeutic agent alone;
(b) the drug delivery composition exhibits reduced occurrence of accumulation in healthy organs and/or tissues of a mammalian patient when compared to therapeutic agent/potential therapeutic agent alone; and
(c) the drug delivery composition exhibits higher accumulation in tumors and/or diseased tissues of a mammalian patient when compared to therapeutic agent/potential therapeutic agent alone.

19. The drug delivery composition of claim 1, wherein the heparosan polymer comprises an activated group on the heparosan polymer to effect the covalent or non-covalent conjugation of the heparosan polymer to the hydrophobic moiety-containing component, and wherein the reactive group is selected from the group consisting of an aldehyde, alkyne, ketone, maleimide, thiol, azide, amino, carbonyl, sulfhydryl, hydrazide, phosphate, sulfate, nitrate, carbonate, ester, squarate, chelator, and combinations thereof.

20. A method for preparing a pharmaceutically active drug delivery composition, the method comprising the step of:
reacting a plurality of components with at least one therapeutic agent and/or at least one potential therapeutic agent, wherein at least a portion of the plurality of components comprises at least one hydrophobic moiety and wherein the reaction occurs under conditions sufficient to effect aggregation of the hydrophobic moieties via hydrophobic interactions to form at least one multimolecular assembly from the plurality of components, and wherein the reaction conditions are also sufficient to entrap or bind the at least one therapeutic/potential therapeutic agent within or to the multimolecular assembly, at least one of the hydrophobic moiety-containing components of the multimolecular assembly having a heparosan polymer attached thereto whereby the at least one heparosan polymer is attached to a surface of the multimolecular assembly, and wherein the heparosan polymer is characterized as being substantially non-antigenic, substantially non-immunogenic, and substantially biologically inert within extracellular compartments of a mammalian patient, being stable in the mammalian bloodstream, and being degraded intracellularly in the mammalian patient.

21. The method of claim 20, wherein the drug delivery composition is the drug delivery composition of any one of claims 2-19.

22. A method for preparing a pharmaceutically active drug delivery composition, the method comprising the steps of:
reacting a plurality of components with at least one therapeutic agent and/or at least one potential therapeutic agent, wherein at least a portion of the plurality of components comprises at least one hydrophobic moiety and wherein the reaction occurs under conditions sufficient to effect aggregation of the hydrophobic moieties via hydrophobic interactions to form at least one multimolecular assembly from the plurality of components, and wherein the reaction conditions are also sufficient to entrap or bind the at least one therapeutic/potential therapeutic agent within or to the multimolecular assembly; and
reacting the at least one multimolecular assembly with at least one heparosan polymer having a hydrophobic moiety-containing component attached thereto, wherein the at least one hydrophobic moiety attached to the heparosan polymer partitions into the at least one multimolecular assembly whereby the at least one heparosan polymer is attached to a surface of the multimolecular assembly, and wherein the heparosan polymer is characterized as being substantially non-antigenic, substantially non-immunogenic, and substantially biologically inert within extracellular compartments of a mammalian patient, being stable in the mammalian bloodstream, and being degraded intracellularly in the mammalian patient.

23. The method of claim 22, wherein the drug delivery composition is the drug delivery composition of any one of claims 2-19.

24. A method, comprising the step of:
administering a therapeutically effective amount of the drug delivery composition of claim 1 to a mammalian patient so as to induce a therapeutic effect in the mammalian patient.

25. The method of claim 24, wherein the therapeutically effective amount of the drug delivery composition is injected into the mammalian patient.

26. The method of claim 24, wherein the drug delivery composition is further defined as the drug delivery composition of any one of claims 2-19.
